(19) United States
(12) Patent Application Publication
(10) Pub. No.: US 2026/0284260 A1
(43) Pub. Date: Sep. 24, 2026

(54) CONJUGATES COMPRISING AN ANTIBACTERIAL AGENT AND METHODS OF USING THEREOF

(71) Applicant: UNIVERSITY OF MASSACHUSETTS, Westborough, MA (US)

(72) Inventor: Jie SONG, Shrewsbury, MA (US)

(21) Appl. No.: 19/575,817

(22) Filed: Mar. 23, 2026

Related U.S. Application Data
(60) Provisional application No. 63/776,107, filed on Mar. 22, 2025.

(51) Int. Cl. A61L 27/34; A61K 47/61; A61L 27/54; A61P 31/04; C08L 5/08

(52) U.S. Cl. CPC A61L 27/34; A61K 47/61; A61L 27/54; A61P 31/04; C08L 5/08; A61L 2300/406

(57) ABSTRACT

Disclosed herein are methods for improving the activity of antibacterial agents against Gram negative bacteria, as well as conjugates comprising an antibacterial agent covalently linked to a metal-chelating polymer (MCP). Also described herein are polymer-drug conjugates that can exhibit potent antimicrobial activity against Gram positive bacteria, Gram negative bacteria, or both Gram positive bacteria and Gram negative bacteria, as well as methods of using thereof.

### CROSS-REFERENCE TO RELATED APPLICATIONS

This application claims benefit of priority of U.S. Provisional Application No. 63/776,107, filed Mar. 22, 2025, which is hereby incorporated by reference in its entirety.

### STATEMENT OF GOVERNMENT SUPPORT

This invention was made with government support under Grant No. R01AR078044 awarded by the National Institutes of Health. The government has certain rights in the invention.

### BACKGROUND

Drug-resistant bacteria are a major public health concern, with Gram-negative bacteria particularly troubling as they are insensitive to many commonly used antibiotics. Exacerbating this problem is the fact that a new class of antibiotics active against Gram-negative bacteria has not been introduced into the clinic since the quinolones in 1968. This void in discovery is not due to a lack of effort; as one example, in 2007 GlaxoSmithKline reported screening ^{~}500,000 synthetic compounds for whole cell activity against Escherichia coli, but no tractable hits were identified.

The difficulty of killing Gram-negative pathogens is largely attributed to the structure of their outer membranes. Gram-negative bacteria possess two cellular membranes, with the outer membrane allowing only very slow passive diffusion of small molecules. Once inside the cell, small molecules are susceptible to efflux pumps; thus, in order to accumulate in Gram-negatives, small molecules must cross the outer membrane at a faster rate than they are pumped out. In order to accumulate to a level sufficient for activity, small molecules typically must cross the outer-membrane via channel proteins called porins, which are narrow β-barrels lined with charged amino acids that serve as selective gateways to entry for many small molecule antibiotics.

For many Gram-negative species, antibiotics enter through general porins such as OmpF, the prototypical porin of E. coli. Although general porins are wider than typical substrate specific channels, most contain a relatively narrow constriction zone, limiting the size of small molecules capable of diffusing through. For example, the constriction zone of OmpF is approximately 7×11 Å, which is believed to restrict passive diffusion of small molecules to an estimated 600 Da.

Central to the problem of Gram-negative antibiotic discovery is a limited understanding of the physicochemical properties that enable small molecule accumulation in Gram-negative bacteria, with current knowledge based largely on retrospective analyses of known antibiotics, and free energy calculations of small molecule permeation across the outer membrane. In 2008 O'Shea and Moser reported that antibiotics effective against Gram-negative pathogens almost always have a molecular weight (MW) less than 600 Da and tend to be very polar as measured by C log D_{7.4}, observations consistent with porin architecture. Retrospective studies by others have reinforced these observations that Gram-negative active compounds tend to be small and highly polar. However, there are a number of antibiotics that meet these polarity and size criteria but are inactive against Gram-negative species, suggesting these properties do not fully encompass the determinants for small molecule accumulation.

Additionally, retrospective analyses are highly skewed by the over-representation of certain drug classes. For example, an analysis by AstraZeneca showed that carboxylic acids are present on up to 40% of Gram-negative active compounds in their collection; however, these carboxylic-acid-containing compounds are almost exclusively β-lactams. While a handful of compound accumulation studies in whole cells have been performed, broad conclusions cannot be drawn from these small data sets (10-20 compounds and all within a single structural class). Perhaps most importantly, the canonical view about the importance of C log D_{7.4} and MW for Gram-negative activity has not led to general strategies to convert Gram-positive-only compounds into broad-spectrum antibiotics. The seminal observation over 50 years ago that derivatizing penicillin G into ampicillin results in broad-spectrum activity has not been generalizable, and important classes of experimental therapeutics and FDA-approved antibiotics have coverage only against Gram-positive organisms despite intensive derivatization efforts.

Antimicrobial resistance among Gram-positive bacterial pathogens, including Staphylococcus aureus, Streptococcus spp. and Enterococcus spp., has also emerged as a major global health challenge, resulting in elevated morbidity, mortality and healthcare costs. Clinically important strains such as methicillin-resistant S. aureus (MRSA), vancomycin-resistant enterococci (VRE) and vancomycin-resistant S. aureus (VRSA) exhibit resistance to multiple front-line agents, thereby limiting effective treatment options and driving the need for new modalities that can overcome established resistance mechanisms.

Therefore, there exists a need for new strategies for improving the efficacy of antibiotics, including antibiotics with activity against Gram-positive bacteria and Gram-negative bacteria. Further, there exists a need for compositions and methods that can sensitize Gram-negative bacteria to a range of antibiotic compounds.

### SUMMARY

Described herein are methods of improving the activity of an antibacterial agent against a Gram-negative bacteria. These methods can comprise conjugating the antibacterial agent to a metal-chelating polymer (MCP).

The MCP can comprise any suitable polymer that can chelate metal ions (e.g., bivalent cations that crosslink the lipopolysaccharide component of the outer membrane of the Gram negative bacteria, such as calcium ions and magnesium ions). In some embodiments, the MCP is a negatively charged polymer. In some embodiments, the MCP can include one or more carboxylate moieties, one or more amine moieties, one or more hydroxy moieties, one or more ether moieties,

In some embodiments, the MCP has a molecular weight of from 4000 Da to 500 kDa, such as a molecular weight of from 50 kDa to 100 kDa.

In some embodiments, the MCP can comprise a synthetic polymer. In some embodiments, the MCP comprises a biopolymer. In certain embodiments, the MCP comprises a polysaccharide.

In some embodiments, the MCP comprises a polymer bearing multiple carboxylic acid moieties, such as a poly(meth)acrylic acid or a copolymer thereof (e.g., poly(acrylic acid-co-maleic acid) (PAM)), a polyamine polymer, such as polyethyleneimine (PEI) or a copolymer thereof, a polymer bearing multiple iminodiacetic acid (IDA) moieties, such as a polymer derived from glycidyl methacrylate (GMA) and then modified to include modified to include IDA moieties, or a polysaccharide, such as chitosan, alginate, pectin, cellulose, glycogen, chitin, starch such as potato starch, glycosaminoglycans such as chondroitin, chondroitin sulfate, dermatan sulfate, hyaluronic acid, and keratan sulfate; aminated dextrans including DEAE-dextran; aminated starch, aminated glycogen, aminated cellulose, aminated pectin, and salts, complexes, derivatives and mixtures thereof.

In some embodiments, the antibacterial agent is covalently bound to the MCP via a non-cleavable linker. In other embodiments, the antibacterial agent is covalently bound to the MCP via a cleavable linker.

In some embodiments, the antibacterial agent is an antibiotic that is indicated for use against a Gram positive bacterial infection. In some embodiments, the antibacterial agent is an antibiotic that is not indicated for use against a Gram negative bacterial infection.

In some embodiments, the antibacterial agent comprises a glycopeptide antibiotic, such as vancomycin, teicoplanin, telavancin, oritavancin, dalbavancin, bleomycin, ramoplanin, or decaplanin. In some embodiments, the antibacterial agent comprises a large beta-lactam antibiotic (i.e., a beta-lactam antibiotic that is not indicated for use against a Gram negative bacterial infection), such as penicillin G or nafcillin/oxacillin. In some embodiments, the antibacterial agent comprises a macrolide antibiotic, such as erythromycin, clarithromycin, azithromycin, roxithromycin, telithromycin, fidaxomicin, oleandomycin, methymycin, or tylosin. In some embodiments, the antibacterial agent comprises a lipopeptide antibiotic, such as daptomycin, polymyxins (colistin and polymyxin B), surfactin, iturin, or fengycin; or a lincosamide antibiotic, such as clindamycin.

In some embodiments, the method further comprises conjugating a second antibacterial agent to the MCP. In some embodiments, the second antibacterial agent comprises an antibiotic that is not indicated for use against both a Gram negative bacterial infection and a Gram positive bacterial infection (e.g., a broad spectrum antibiotic), and wherein the second antibacterial agent is covalently bound to the MCP via a cleavable linker. In some embodiments, the second antibacterial agent comprises doxycycline, minocycline, an aminoglycoside antibiotic (other than streptomycin), ampicillin, amoxicillin/clavulanic acid (Augmentin), azithromycin, a carbapenem antibiotic (e.g., imipenem), piperacillin/tazobactam, a quinolone antibiotic (e.g., ciprofloxacin), a tetracycline antibiotic (other than sarecycline), chloramphenicol, ticarcillin, trimethoprim/sulfamethoxazole (Bactrim), ofloxacin, or a combination thereof.

In some embodiments, conjugating the antibacterial agent to the MCP affords an antibacterial agent-MCP conjugate comprising one or more covalently modified monomers defined by Formula I

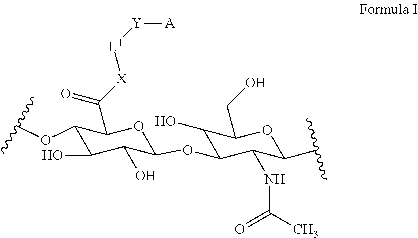

wherein A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, the linking group comprises a moiety formed by the chemical reaction of two click motifs, and wherein conjugating the antibacterial agent to the MCP comprises performing a click reaction between an MCP bearing a first click motif and an antibacterial agent bearing a second click motif.

In some embodiments, conjugating the antibacterial agent to the MCP affords an antibacterial agent-MCP conjugate comprising a random copolymer defined by Formula II below

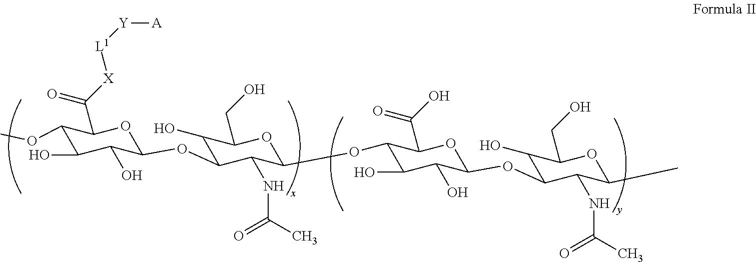

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, conjugating the antibacterial agent to the MCP affords an antibacterial agent-MCP conjugate comprising a random copolymer defined by Formula III below

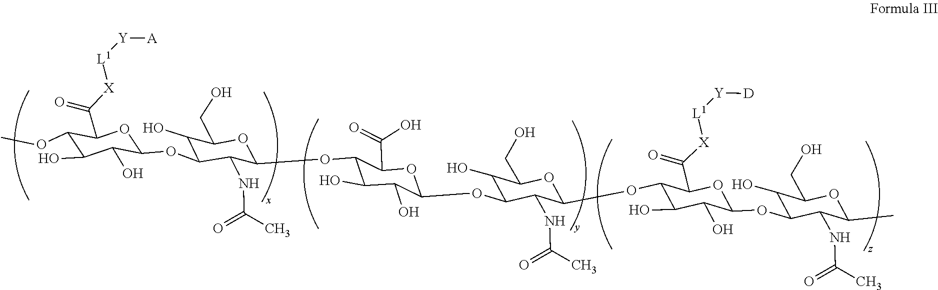

wherein x, y, and z are each independently integers from 1 to 2500, wherein x+y+z is no more than 2500, and wherein x, y, and z represent the relative portion of each monomer within the random copolymer; A is a first antibacterial agent; D is a second antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In certain embodiments, the first antibacterial agent comprises an antibacterial agent that targets bacterial cell walls, and the second antibacterial agent comprises an antibacterial agent that targets the cytoplasmic membrane of bacterial cells. In certain embodiments, the antibacterial agent that targets bacterial cell walls is chosen from a β-lactam antibiotic, a glycopeptide antibiotic, a lipoglycopeptide antibiotic, and bacitracin, optionally comprising a penicillin, a cephalosporin, a carbapenem, vancomycin, teicoplanin, dalbavancin, oritavancin, telavancin, or a pharmaceutically acceptable salt thereof. In certain embodiments, the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof. These conjugates can exhibit strong antibacterial activity against gram positive bacteria.

Also provided herein are polymer-drug conjugate comprising an antibacterial agent conjugated to a polymer. In some embodiments, these polymer-drug conjugates do not require cleavage and/or degradation to exhibit antibacterial activity. In some embodiments, the polymer-drug conjugate exhibits a molecular weight of at least 5,000 Da (e.g., at least 10,000 Da, at least 15,000 Da, at least 20,000 Da, at least 25,000 Da, at least 50,000 Da, at least 75,000 Da, or at least 100,000 Da) when active intracellularly within a target bacterial cell.

In some embodiments, the polymer-drug conjugate interacts with at least two different structural components of the target bacterial cell to produce cytotoxic activity.

In some embodiments, the target bacterial cell comprises a Gram positive bacteria, and the polymer-drug conjugate interacts with a cell wall of the Gram positive bacteria and a cytoplasmic membrane of the Gram positive bacteria.

In some of these embodiments, the polymer-drug conjugate is amphiphilic. In some embodiments, the polymer-drug conjugate comprises a hydrophobic antibacterial agent conjugated to a hydrophilic polymer. In certain embodiments, the hydrophobic antibacterial agent comprises an antibacterial agent that targets bacterial cell walls. For example, the hydrophobic antibacterial agent can be chosen from a β-lactam antibiotic, a glycopeptide antibiotic, a lipoglycopeptide antibiotic, and bacitracin, optionally wherein the antibacterial agent that targets bacterial cell walls is a penicillin, a cephalosporin, a carbapenem, vancomycin, teicoplanin, dalbavancin, oritavancin, telavancin, or any combination thereof.

In some embodiments, the polymer-drug conjugate further comprises a second antibacterial agent conjugated to the polymer, wherein the second antibacterial agent comprises an agent that targets the cytoplasmic membrane of bacterial cells. In certain embodiments, the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

In certain embodiments, the hydrophilic polymer comprises a polysaccharide, such as hyaluronic acid.

In some embodiments, the polymer-drug conjugate comprises one or more covalently modified monomers defined by Formula I

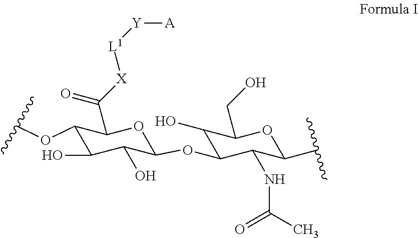

wherein A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S. In certain embodiments, the linking group comprises a moiety formed by the chemical reaction of two click motifs.

In some embodiments, the polymer-drug conjugate comprises a random copolymer defined by Formula II below

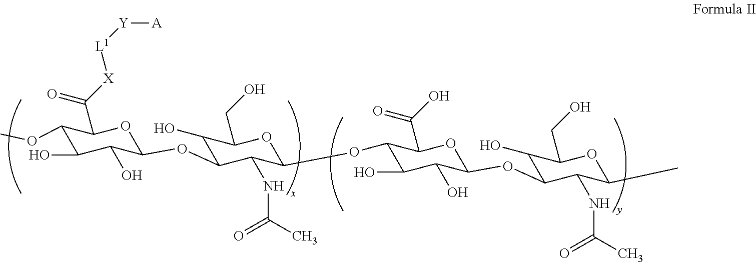

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)X—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, the polymer-drug conjugate comprises a random copolymer defined by Formula III below

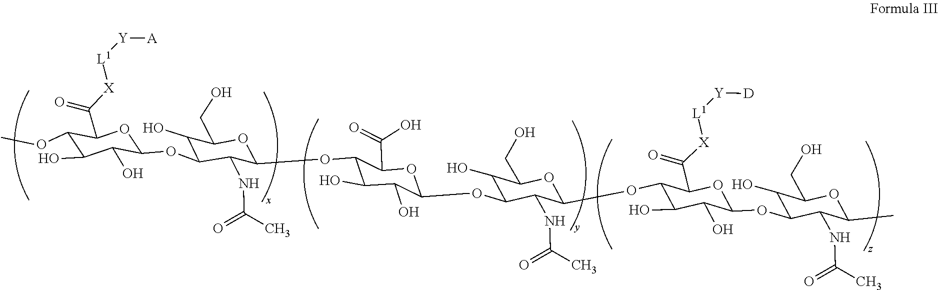

wherein x, y, and z are each independently integers from 1 to 2500, wherein x+y+z is no more than 2500, and wherein x, y, and z represent the relative portion of each monomer within the random copolymer; A is a first antibacterial agent; D is a second antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In certain embodiments, the first antibacterial agent comprises an antibacterial agent that targets bacterial cell walls, and the second antibacterial agent comprises an antibacterial agent that targets the cytoplasmic membrane of bacterial cells. In certain embodiments, the antibacterial agent that targets bacterial cell walls is chosen from a β-lactam antibiotic, a glycopeptide antibiotic, a lipoglycopeptide antibiotic, and bacitracin, optionally comprising a penicillin, a cephalosporin, a carbapenem, vancomycin, teicoplanin, dalbavancin, oritavancin, telavancin, or a pharmaceutically acceptable salt thereof. In certain embodiments, the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

In other embodiments, the target bacterial cell comprises a Gram negative bacteria, and the polymer-drug conjugate interacts with an outer membrane of the Gram negative bacteria and a cell wall of the Gram negative bacteria. In some of these embodiments, the polymer-drug conjugate is an antibacterial agent-MCP conjugate comprising an antibacterial agent conjugated to a metal-chelating polymer (MCP).

The MCP can comprise any suitable polymer that can chelate metal ions (e.g., bivalent cations that crosslink the lipopolysaccharide component of the outer membrane of the Gram negative bacteria, such as calcium ions and magnesium ions). In some embodiments, the MCP is a negatively charged polymer. In some embodiments, the MCP can include one or more carboxylate moieties, one or more amine moieties, one or more hydroxy moieties, one or more ether moieties,

In some embodiments, the MCP has a molecular weight of from 4000 Da to 500 kDa, such as a molecular weight of from 50 kDa to 100 kDa.

In some embodiments, the MCP can comprise a synthetic polymer. In some embodiments, the MCP comprises a biopolymer. In certain embodiments, the MCP comprises a polysaccharide.

In some embodiments, the MCP comprises a polymer bearing multiple carboxylic acid moieties, such as a poly(meth)acrylic acid or a copolymer thereof (e.g., poly(acrylic acid-co-maleic acid) (PAM)), a polyamine polymer, such as polyethyleneimine (PEI) or a copolymer thereof, a polymer bearing multiple iminodiacetic acid (IDA) moieties, such as a polymer derived from glycidyl methacrylate (GMA) and then modified to include modified to include IDA moieties, or a polysaccharide, such as chitosan, alginate, pectin, cellulose, glycogen, chitin, starch such as potato starch, glycosaminoglycans such as chondroitin, chondroitin sulfate, dermatan sulfate, hyaluronic acid, and keratan sulfate; aminated dextrans including DEAE-dextran; aminated starch, aminated glycogen, aminated cellulose, aminated pectin, and salts, complexes, derivatives and mixtures thereof.

In some embodiments, the antibacterial agent is covalently bound to the MCP via a non-cleavable linker. In other embodiments, the antibacterial agent is covalently bound to the MCP via a cleavable linker.

In some embodiments, the antibacterial agent is an antibiotic that is indicated for use against a Gram positive bacterial infection. In some embodiments, the antibacterial agent is an antibiotic that is not indicated for use against a Gram negative bacterial infection.

In some embodiments, the antibacterial agent comprises a glycopeptide antibiotic, such as vancomycin, teicoplanin, telavancin, oritavancin, dalbavancin, bleomycin, ramoplanin, or decaplanin. In some embodiments, the antibacterial agent comprises a large beta-lactam antibiotic (i.e., a beta-lactam antibiotic that is not indicated for use against a Gram negative bacterial infection), such as penicillin G or nafcillin/oxacillin. In some embodiments, the antibacterial agent comprises a macrolide antibiotic, such as erythromycin, clarithromycin, azithromycin, roxithromycin, telithromycin, fidaxomicin, oleandomycin, methymycin, or tylosin. In some embodiments, the antibacterial agent comprises a lipopeptide antibiotic, such as daptomycin, polymyxins (colistin and polymyxin B), surfactin, iturin, or fengycin; or a lincosamide antibiotic, such as clindamycin.

In some embodiments, the method further comprises conjugating a second antibacterial agent to the MCP. In some embodiments, the second antibacterial agent comprises an antibiotic that is not indicated for use against both a Gram negative bacterial infection and a Gram positive bacterial infection (e.g., a broad spectrum antibiotic), and wherein the second antibacterial agent is covalently bound to the MCP optionally via a cleavable linker. In some embodiments, the second antibacterial agent comprises doxycycline, minocycline, an aminoglycoside antibiotic (other than streptomycin), ampicillin, amoxicillin/clavulanic acid (Augmentin), azithromycin, a carbapenem antibiotic (e.g., imipenem), piperacillin/tazobactam, a quinolone antibiotic (e.g., ciprofloxacin), a tetracycline antibiotic (other than sarecycline), chloramphenicol, ticarcillin, trimethoprim/sulfamethoxazole (Bactrim), ofloxacin, or a combination thereof.

In some embodiments, the antibacterial agent-MCP conjugate can comprise one or more covalently modified monomers defined by Formula I

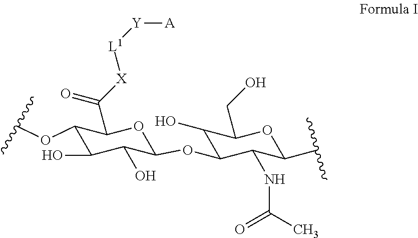

wherein A is an antibacterial agent; Y is absent, or is chosen from —O(C═O)—, —C(═O)—, —S(O)—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, the linking group comprises a moiety formed by the chemical reaction of two click motifs, and wherein conjugating the antibacterial agent to the MCP comprises performing a click reaction between an MCP bearing a first click motif and an antibacterial agent bearing a second click motif.

In some embodiments, the antibacterial agent-MCP conjugate can comprise a random copolymer defined by Formula II below

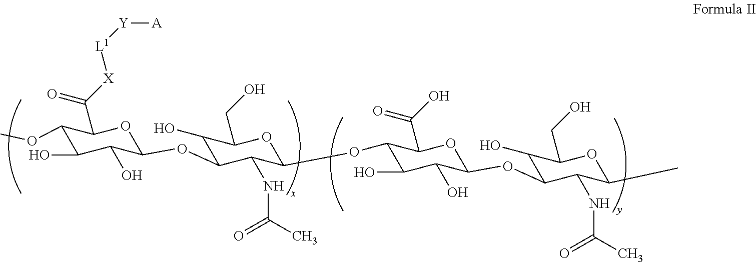

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

Also provided herein are methods of treating or preventing a Gram negative bacterial infection in a subject. These methods can comprise administering to the subject a therapeutically or prophylactically effective amount of a polymer-drug conjugate described herein.

In some embodiments, the Gram negative bacterial infection is caused by a bacteria of one of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella.

Also provided herein are methods of treating or preventing a Gram positive bacterial infection in a subject. These methods can comprise administering to the subject a therapeutically or prophylactically effective amount of a polymer-drug conjugate described herein.

Also provided herein are methods of treating or preventing a bacterial infection in a subject comprising a small colony variant. These methods can comprise administering to the subject a therapeutically or prophylactically effective amount of a polymer-drug conjugate described herein. In some examples, the small colony variant comprises Staphylococcus aureus.

Also provided herein are methods of providing broad spectrum antibiotic therapy to a subject in need thereof to treat or prevent bacterial infection. These methods can comprise administering to the subject a therapeutically or prophylactically effective amount of a polymer-drug conjugate described herein.

Also provided herein are methods of sensitizing a Gram negative bacteria towards an antimicrobial agent. These methods can comprise contacting the Gram negative bacteria with a polymer-drug conjugate described herein.

In some embodiments, the Gram negative bacterial infection is caused by a bacteria of one of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella.

Also provided herein are medical devices comprising a coating comprising a polymer-drug conjugate described herein.

In some embodiments, the medical device comprises an implantable (e.g., indwelling) medical device. In some embodiments, the medical device is formed at least in part from a metal, a ceramic, a polymer, or a combination thereof.

In some embodiments, the medical device comprises an orthopedic implant or device, a catheter, a suture (resorbable or non-resorbable), a cardiovascular device (e.g., a pacemaker, a stent), an endotracheal tube, a tracheotomy tube, chest tube, a vascular catheter, an urinary catheter, a nephrostomy tube, a biliary stent, a peritoneal catheter, an epidural catheter, a central nervous system catheter, a prosthetic valve, a gastric tube, an intestinal tube, or a drug-delivery implant.

### DESCRIPTION OF DRAWINGS

FIGS. 1A-1C. ¹H-NMR spectra of (FIG. 1A) vancomycin vs. HA-Vanco conjugate in D₂O, (FIG. 1B) vancomycin vs. PAA-Vanco conjugates with R=0.04, 0.06, 0.1 and 0.5 in D₂O, (FIG. 1C) vancomycin vs. filtrates of PAA-Vanco aq. suspension with R=0.1 and 0.5 in DMSO-d6.

FIGS. 2A-2B. Degree of cleavage of HA-Vanco and HA by 10K CFUs of S. aureus (FIG. 2A) and E. coli (FIG. 2B) overnight determined by GPC.

FIGS. 3A-3B. Antimicrobial properties of uncleaved, pre-cleaved HA-Vanco, low molecular weight (LMW) HA-Vanco and vancomycin against S. aureus (FIG. 3A) and E. coli (FIG. 3B). LMW HA-Vanco is prepared from HA with Mw of 10-25 kD. Concentration of all samples, except for untreated control, was kept at 250 μM of vancomycin residues conjugated or 250 μM free vancomycin.

FIGS. 4A-4C. A₆₃₀ₙₘ of (FIG. 4A) E. coli and (FIG. 4B) S. aureus suspension as a function of HA-Vanco concentration after 24-h incubation. Both curves display two-staged inhibitions, indicated by dash and solid fitting curves. Minimum inhibitory concentration (MIC) of HA-Vanco vs. vancomycin against S. aureus and E. coli are shown in FIG. 4C. The MIC₅₀ of HA-Vanco within the 1^{st} stage of A630 nm inhibition is also shown.

FIG. 4D. TEM micrographs showing that HA-Vanco conjugate, but not vancomycin, severely disrupts E. coli cell envelop integrity, resulting in severely distorted and swollen cell morphology. Scale Bars: 05 microns.

FIGS. 5A-5D. Overlay of bright field image and DAPI staining showing the morphologies of E. coli after 3-hr co-culture with (FIG. 5A) PBS, (FIG. 5B) HA, (FIG. 5C) vancomycin, and (FIG. 5D) HA-Vanco. The concentration of molecules added was fixed at 50 μM in the E. coli culture.

FIG. 5E. HA-Vanco more effectively inhibits E. coli than EDTA, and this activity can be abolished by Mg²⁺ supplement.

FIG. 6A. TEM showing that the macromolecular HA-Vanco conjugate but not vancomycin rapidly destructs cytoplasmic membrane of S. aureus via mesosome formation (arrows).

FIG. 6B. HA-Vanco conjugate more effectively kills small colony variant (SCV) of S. aureus while extended vancomycin treatment selects for SCV.

FIGS. 7A-7C. Proposed antibacterial mechanism of HA-Vanco against (FIG. 7A) S. aureus and (FIG. 7B) E. coli, and potential sensitization of other antibiotics (FIG. 7C). (FIG. 7A) The vancomycin residues on the HA-Vanco conjugate binds with D-Ala-D-Ala in the outer peptidoglycan layer of S. aureus upon contact to inhibit the cell wall synthesis. The conjugate then can interact with and disrupt the cytoplasmic membrane of the bacterial cell. These binding events occurred efficiently, resulting in the rapid killing of the Gram positive bacteria without the need for the macromolecule being cleaved by hyaluronate lyase first. (FIG. 7B) For E. coli, the anionic HA-Vanco effectively chelates the bivalent cations that crosslink the lipopolysaccharide component of the outer membrane of the Gram negative bacteria, thereby destabilizing/permeating the outer membrane for HA-Vanco to enter. Once getting across the outer membrane, the vancomycin residues of the HA-Vanco can bind to D-Ala-D-Ala of the peptidoglycan layer to disrupt the cell wall synthesis. (FIG. 7C) Destabilization of the E. coli outer membrane with HA-Vanco could sensitize other antibiotics synergistically delivered by facilitating their entry across the leaky outer membrane.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

“Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

“Effective amount” or “therapeutically effective amount”, as used herein, refers to an amount of polymer-drug conjugate effective to alleviate, delay onset of, or prevent one or more symptoms of a disease or disorder being treated by the active agent, and/or an amount of polymer-drug conjugate effective to produce a desired diagnostic signal.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer” as used herein, generally refers to a polymer that will degrade or erode by enzymatic action or hydrolysis under physiologic conditions to smaller units or chemical species that are capable of being metabolized, eliminated, or excreted by the subject. The degradation time is a function of polymer composition, morphology, such as porosity, particle dimensions, and environment.

“Molecular weight” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Pharmaceutically Acceptable”, as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

‘Polysaccharides’ are relatively complex carbohydrates. They are polymers made up of many monosaccharides joined together by glycosidic bonds. They are therefore very large, often branched, macromolecules. They tend to be amorphous, insoluble in water, and have no sweet taste. When all the monosaccharides in a polysaccharide are the same type the polysaccharide is called a homopolysaccharide, but when more than one type of monosaccharide is present they are called heteropolysaccharides. Examples include storage polysaccharides such as starch and glycogen and structural polysaccharides such as cellulose and chitin. Polysaccharides have a general formula of Cₙ(H₂O)ₙ₋₁ where n is usually a large number between 200 and 2500. Considering that the repeating units in the polymer backbone are often six-carbon monosaccharides, the general formula can also be represented as (C₆H₁₀O₅)ₙ where n={40 . . . 3000}.

The term “hyaluronic acid polymer” refers to a polymer comprising repeat disaccharide subunits of hyaluronan, where the repeat units may be derivatized at one or more positions of the D-glucuronic acid and/or the D-N-acetylglucosamine unit of the disaccharide repeat subunit A hyaluronic acid polymer is meant to encompass hyaluronic acid (also referred to as hyaluronan), derivatized hyaluronic acid, salts forms, hyaluronic acid linker complexes, and hyaluronic acid conjugates. The terms “hyaluronic acid derivative” or “derivatized hyaluronic acid” or “modified hyaluronic acid” refers to a hyaluronic acid polymer which has been derivatized by reaction with, e g, one or more chemical moieties.

Ranges of values defined herein include all values within the range as well as all sub-ranges within the range. For example, if the range is defined as an integer from 0 to 10, the range encompasses all integers within the range and any and all subranges within the range, e.g., 1-10, 1-6, 2-8, 3-7, 3-9, etc.

### Conjugates and Methods

Described herein are methods of improving the activity of an antibacterial agent against a Gram-negative bacteria. These methods can comprise conjugating the antibacterial agent to a metal-chelating polymer (MCP).

The MCP can comprise any suitable polymer that can chelate metal ions (e.g., bivalent cations that crosslink the lipopolysaccharide component of the outer membrane of the Gram negative bacteria, such as calcium ions and magnesium ions). In some embodiments, the MCP is a negatively charged polymer. In some embodiments, the MCP can include one or more carboxylate moieties, one or more amine moieties, one or more hydroxy moieties, one or more ether moieties,

In some embodiments, the MCP has a molecular weight of from 4000 Da to 500 kDa, such as a molecular weight of from 50 kDa to 100 kDa.

In some embodiments, the MCP can comprise a synthetic polymer. In some embodiments, the MCP comprises a biopolymer. In certain embodiments, the MCP comprises a polysaccharide.

In some embodiments, the MCP comprises a polymer bearing multiple carboxylic acid moieties, such as a poly(meth)acrylic acid or a copolymer thereof (e.g., poly(acrylic acid-co-maleic acid) (PAM)), a polyamine polymer, such as polyethyleneimine (PEI) or a copolymer thereof, a polymer bearing multiple iminodiacetic acid (IDA) moieties, such as a polymer derived from glycidyl methacrylate (GMA) and then modified to include modified to include IDA moieties, or a polysaccharide, such as chitosan, alginate, pectin, cellulose, glycogen, chitin, starch such as potato starch, glycosaminoglycans such as chondroitin, chondroitin sulfate, dermatan sulfate, hyaluronic acid, and keratan sulfate; aminated dextrans including DEAE-dextran; aminated starch, aminated glycogen, aminated cellulose, aminated pectin, and salts, complexes, derivatives and mixtures thereof.

In some embodiments, the antibacterial agent is covalently bound to the MCP via a non-cleavable linker. In other embodiments, the antibacterial agent is covalently bound to the MCP via a cleavable linker.

In some embodiments, the antibacterial agent is an antibiotic that is indicated for use against a Gram positive bacterial infection. In some embodiments, the antibacterial agent is an antibiotic that is not indicated for use against a Gram negative bacterial infection.

In some embodiments, the antibacterial agent comprises a glycopeptide antibiotic, such as vancomycin, teicoplanin, telavancin, oritavancin, dalbavancin, bleomycin, ramoplanin, or decaplanin. In some embodiments, the antibacterial agent comprises a large beta-lactam antibiotic (i.e., a beta-lactam antibiotic that is not indicated for use against a Gram negative bacterial infection), such as penicillin G or nafcillin/oxacillin. In some embodiments, the antibacterial agent comprises a macrolide antibiotic, such as erythromycin, clarithromycin, azithromycin, roxithromycin, telithromycin, fidaxomicin, oleandomycin, methymycin, or tylosin. In some embodiments, the antibacterial agent comprises a lipopeptide antibiotic, such as daptomycin, polymyxins (colistin and polymyxin B), surfactin, iturin, or fengycin; or a lincosamide antibiotic, such as clindamycin.

In some embodiments, the method further comprises conjugating a second antibacterial agent to the MCP. In some embodiments, the second antibacterial agent comprises an antibiotic that is not indicated for use against both a Gram negative bacterial infection and a Gram positive bacterial infection (e.g., a broad spectrum antibiotic), and wherein the second antibacterial agent is covalently bound to the MCP via a cleavable linker. In some embodiments, the second antibacterial agent comprises doxycycline, minocycline, an aminoglycoside antibiotic (other than streptomycin), ampicillin, amoxicillin/clavulanic acid (Augmentin), azithromycin, a carbapenem antibiotic (e.g., imipenem), piperacillin/tazobactam, a quinolone antibiotic (e.g., ciprofloxacin), a tetracycline antibiotic (other than sarecycline), chloramphenicol, ticarcillin, trimethoprim/sulfamethoxazole (Bactrim), ofloxacin, or a combination thereof.

In some embodiments, conjugating the antibacterial agent to the MCP affords an antibacterial agent-MCP conjugate comprising one or more covalently modified monomers defined by Formula I

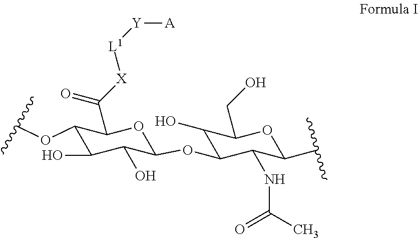

wherein A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, the linking group comprises a moiety formed by the chemical reaction of two click motifs, and wherein conjugating the antibacterial agent to the MCP comprises performing a click reaction between an MCP bearing a first click motif and an antibacterial agent bearing a second click motif.

In some embodiments, conjugating the antibacterial agent to the MCP affords an antibacterial agent-MCP conjugate comprising a random copolymer defined by Formula II below

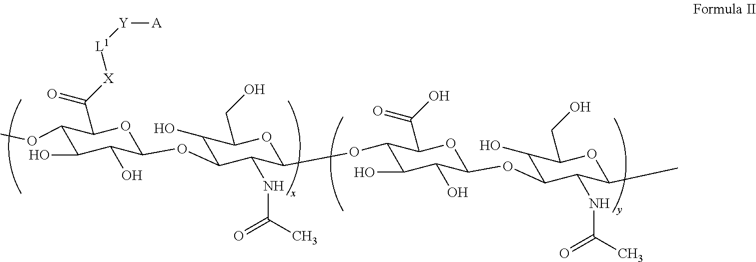

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments of Formula II, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula II, x can be from 1 to 2400 (e.g., from 1 to 1500, from 1 to 1000, from 25 to 1000, or from 25 to 500).

In some embodiments of Formula II, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula II, y can be from 10 to 2400 (e.g., from 10 to 1500, from 10 to 1000, from 25 to 1000, or from 25 to 500).

In some embodiments of Formula II, x+y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x+y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x+y can range from any of the minimum values described above to any of the maximum values described above. For example, in some embodiments of Formula II, x+y can be from 10 to 2400 (e.g., from 10 to 1500, from 10 to 1000, from 25 to 1000, or from 25 to 500).

In some embodiments, conjugating the antibacterial agent to the MCP affords an antibacterial agent-MCP conjugate comprising a random copolymer defined by Formula III below

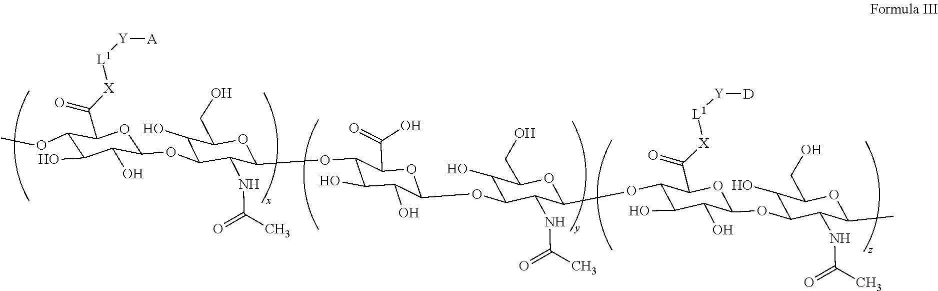

wherein x, y, and z are each independently integers from 1 to 2500, wherein x+y+z is no more than 2500, and wherein x, y, and z represent the relative portion of each monomer within the random copolymer; A is a first antibacterial agent; D is a second antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments of Formula III, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula III, x can be from 1 to 2400 (e.g., from 1 to 1500, from 1 to 1000, from 25 to 1000, or from 25 to 500).

In some embodiments of Formula III, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula III, y can be from 10 to 2400 (e.g., from 10 to 1500, from 10 to 1000, from 25 to 1000, or from 25 to 500).

In some embodiments of Formula III, z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

z can range from any of the minimum values described above to any of the maximum values described above. For example, in some embodiments of Formula III, z can be from 10 to 2400 (e.g., from 10 to 1500, from 10 to 1000, from 25 to 1000, or from 25 to 500).

In some embodiments of Formula III, x+y+z can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula III, x+y+z can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x+y+z can range from any of the minimum values described above to any of the maximum values described above. For example, in some embodiments of Formula III, x+y+z can be from 10 to 2400 (e.g., from 10 to 1500, from 10 to 1000, from 25 to 1000, or from 25 to 500).

In certain embodiments, the first antibacterial agent comprises an antibacterial agent that targets bacterial cell walls, and the second antibacterial agent comprises an antibacterial agent that targets the cytoplasmic membrane of bacterial cells. In certain embodiments, the antibacterial agent that targets bacterial cell walls is chosen from a β-lactam antibiotic, a glycopeptide antibiotic, a lipoglycopeptide antibiotic, and bacitracin, optionally comprising a penicillin, a cephalosporin, a carbapenem, vancomycin, teicoplanin, dalbavancin, oritavancin, telavancin, or a pharmaceutically acceptable salt thereof. In certain embodiments, the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof. These conjugates can exhibit strong antibacterial activity against gram positive bacteria.

Also provided herein are polymer-drug conjugate comprising an antibacterial agent conjugated to a polymer. In some embodiments, these polymer-drug conjugates do not require cleavage and/or degradation to exhibit antibacterial activity. In some embodiments, the polymer-drug conjugate exhibits a molecular weight of at least 5,000 Da (e.g., at least 10,000 Da, at least 15,000 Da, at least 20,000 Da, at least 25,000 Da, at least 50,000 Da, at least 75,000 Da, or at least 100,000 Da) when active intracellularly within a target bacterial cell.

In some embodiments, the polymer-drug conjugate interacts with at least two different structural components of the target bacterial cell to produce cytotoxic activity.

In some embodiments, the target bacterial cell comprises a Gram positive bacteria, and the polymer-drug conjugate interacts with a cell wall of the Gram positive bacteria and a cytoplasmic membrane of the Gram positive bacteria.

In some of these embodiments, the polymer-drug conjugate is amphiphilic. In some embodiments, the polymer-drug conjugate comprises a hydrophobic antibacterial agent conjugated to a hydrophilic polymer. In certain embodiments, the hydrophobic antibacterial agent comprises an antibacterial agent that targets bacterial cell walls. For example, the hydrophobic antibacterial agent can be chosen from a β-lactam antibiotic, a glycopeptide antibiotic, a lipoglycopeptide antibiotic, and bacitracin, optionally wherein the antibacterial agent that targets bacterial cell walls is a penicillin, a cephalosporin, a carbapenem, vancomycin, teicoplanin, dalbavancin, oritavancin, telavancin, or any combination thereof.

In some embodiments, the polymer-drug conjugate further comprises a second antibacterial agent conjugated to the polymer, wherein the second antibacterial agent comprises an agent that targets the cytoplasmic membrane of bacterial cells. In certain embodiments, the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

In certain embodiments, the hydrophilic polymer comprises a polysaccharide, such as hyaluronic acid.

In some embodiments, the polymer-drug conjugate comprises one or more covalently modified monomers defined by Formula I

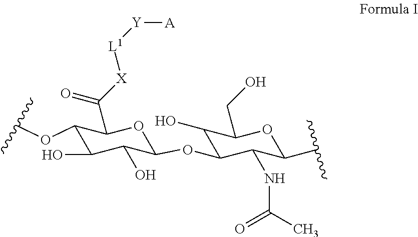

wherein A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S. In certain embodiments, the linking group comprises a moiety formed by the chemical reaction of two click motifs.

In some embodiments, the polymer-drug conjugate comprises a random copolymer defined by Formula II below

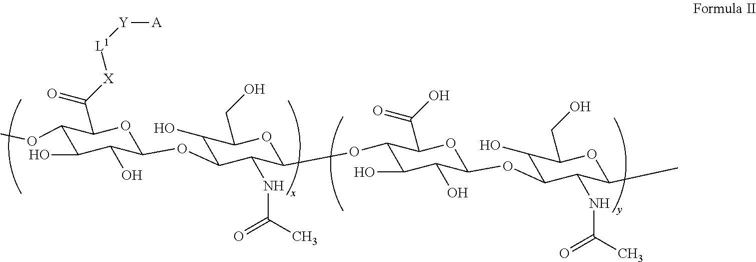

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)X—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, the polymer-drug conjugate comprises a random copolymer defined by Formula III below

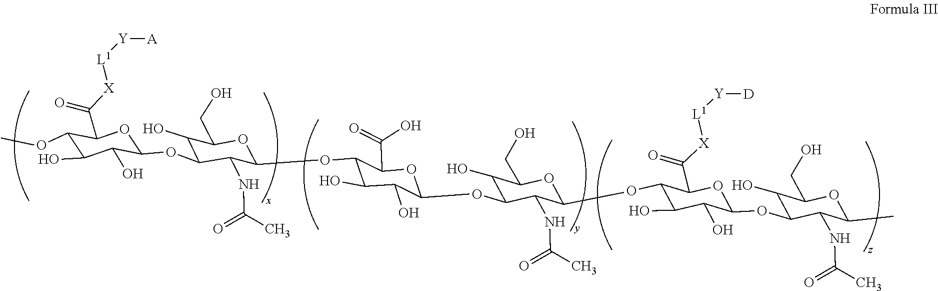

wherein x, y, and z are each independently integers from 1 to 2500, wherein x+y+z is no more than 2500, and wherein x, y, and z represent the relative portion of each monomer within the random copolymer; A is a first antibacterial agent; D is a second antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In certain embodiments, the first antibacterial agent comprises an antibacterial agent that targets bacterial cell walls, and the second antibacterial agent comprises an antibacterial agent that targets the cytoplasmic membrane of bacterial cells. In certain embodiments, the antibacterial agent that targets bacterial cell walls is chosen from a β-lactam antibiotic, a glycopeptide antibiotic, a lipoglycopeptide antibiotic, and bacitracin, optionally comprising a penicillin, a cephalosporin, a carbapenem, vancomycin, teicoplanin, dalbavancin, oritavancin, telavancin, or a pharmaceutically acceptable salt thereof. In certain embodiments, the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

In other embodiments, the target bacterial cell comprises a Gram negative bacteria, and the polymer-drug conjugate interacts with an outer membrane of the Gram negative bacteria and a cell wall of the Gram negative bacteria. In some of these embodiments, the polymer-drug conjugate is an antibacterial agent-MCP conjugate comprising an antibacterial agent conjugated to a metal-chelating polymer (MCP).

The MCP can comprise any suitable polymer that can chelate metal ions (e.g., bivalent cations that crosslink the lipopolysaccharide component of the outer membrane of the Gram negative bacteria, such as calcium ions and magnesium ions). In some embodiments, the MCP is a negatively charged polymer. In some embodiments, the MCP can include one or more carboxylate moieties, one or more amine moieties, one or more hydroxy moieties, one or more ether moieties,

In some embodiments, the MCP has a molecular weight of from 4000 Da to 500 kDa, such as a molecular weight of from 50 kDa to 100 kDa.

In some embodiments, the MCP can comprise a synthetic polymer. In some embodiments, the MCP comprises a biopolymer. In certain embodiments, the MCP comprises a polysaccharide.

In some embodiments, the MCP comprises a polymer bearing multiple carboxylic acid moieties, such as a poly(meth)acrylic acid or a copolymer thereof (e.g., poly(acrylic acid-co-maleic acid) (PAM)), a polyamine polymer, such as polyethyleneimine (PEI) or a copolymer thereof, a polymer bearing multiple iminodiacetic acid (IDA) moieties, such as a polymer derived from glycidyl methacrylate (GMA) and then modified to include modified to include IDA moieties, or a polysaccharide, such as chitosan, alginate, pectin, cellulose, glycogen, chitin, starch such as potato starch, glycosaminoglycans such as chondroitin, chondroitin sulfate, dermatan sulfate, hyaluronic acid, and keratan sulfate; aminated dextrans including DEAE-dextran; aminated starch, aminated glycogen, aminated cellulose, aminated pectin, and salts, complexes, derivatives and mixtures thereof.

In some embodiments, the antibacterial agent is covalently bound to the MCP via a non-cleavable linker. In other embodiments, the antibacterial agent is covalently bound to the MCP via a cleavable linker.

In some embodiments, the antibacterial agent is an antibiotic that is indicated for use against a Gram positive bacterial infection. In some embodiments, the antibacterial agent is an antibiotic that is not indicated for use against a Gram negative bacterial infection.

In some embodiments, the antibacterial agent comprises a glycopeptide antibiotic, such as vancomycin, teicoplanin, telavancin, oritavancin, dalbavancin, bleomycin, ramoplanin, or decaplanin. In some embodiments, the antibacterial agent comprises a large beta-lactam antibiotic (i.e., a beta-lactam antibiotic that is not indicated for use against a Gram negative bacterial infection), such as penicillin G or nafcillin/oxacillin. In some embodiments, the antibacterial agent comprises a macrolide antibiotic, such as erythromycin, clarithromycin, azithromycin, roxithromycin, telithromycin, fidaxomicin, oleandomycin, methymycin, or tylosin. In some embodiments, the antibacterial agent comprises a lipopeptide antibiotic, such as daptomycin, polymyxins (colistin and polymyxin B), surfactin, iturin, or fengycin; or a lincosamide antibiotic, such as clindamycin.

In some embodiments, the method further comprises conjugating a second antibacterial agent to the MCP. In some embodiments, the second antibacterial agent comprises an antibiotic that is not indicated for use against both a Gram negative bacterial infection and a Gram positive bacterial infection (e.g., a broad spectrum antibiotic), and wherein the second antibacterial agent is covalently bound to the MCP via a cleavable linker. In some embodiments, the second antibacterial agent comprises doxycycline, minocycline, an aminoglycoside antibiotic (other than streptomycin), ampicillin, amoxicillin/clavulanic acid (Augmentin), azithromycin, a carbapenem antibiotic (e.g., imipenem), piperacillin/tazobactam, a quinolone antibiotic (e.g., ciprofloxacin), a tetracycline antibiotic (other than sarecycline), chloramphenicol, ticarcillin, trimethoprim/sulfamethoxazole (Bactrim), ofloxacin, or a combination thereof.

In some embodiments, the antibacterial agent-MCP conjugate can comprise one or more covalently modified monomers defined by Formula I

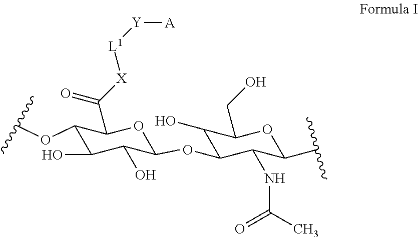

wherein A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments, the linking group comprises a moiety formed by the chemical reaction of two click motifs, and wherein conjugating the antibacterial agent to the MCP comprises performing a click reaction between an MCP bearing a first click motif and an antibacterial agent bearing a second click motif.

In some embodiments, the antibacterial agent-MCP conjugate can comprise a random copolymer defined by Formula II below

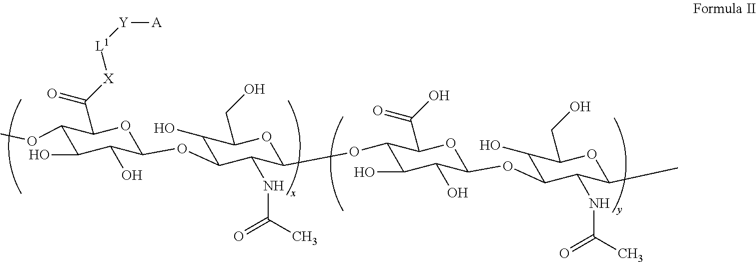

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments of Formula II, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula II, x can be from 1 to 2400 (e.g., from 1 to 1500, from 1 to 1000, from 25 to 1000, or from 25 to 500).

In some embodiments of Formula II, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula II, y can be from 10 to 2400 (e.g., from 10 to 1500, from 10 to 1000, from 25 to 1000, or from 25 to 500).

In some embodiments of Formula II, x+y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x+y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x+y can range from any of the minimum values described above to any of the maximum values described above. For example, in some embodiments of Formula II, x+y can be from 10 to 2400 (e.g., from 10 to 1500, from 10 to 1000, from 25 to 1000, or from 25 to 500).

In some embodiments, the antibacterial agent-MCP conjugate can comprise a random copolymer defined by Formula II below

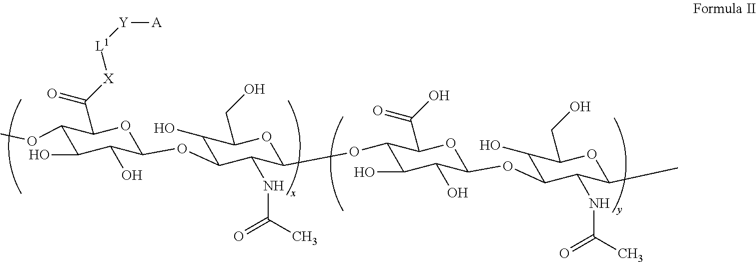

wherein x and y are each independently integers from 1 to 2500, wherein x+y is no more than 2500, and wherein x and y represent the relative portion of each monomer within the random copolymer; A is an antibacterial agent; Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—; L¹ is absent, or is a linking group; and X is absent, or is chosen from NH, O, or S.

In some embodiments of Formula II, x can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x can range from any of the minimum values described above to any of the maximum values described above. For example, in some embodiments of Formula II, x can be from 1 to 2400 (e.g., from 1 to 1500, from 1 to 1000, from 25 to 1000, or from 25 to 500).

In some embodiments of Formula II, y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

y can range from any of the minimum values described above to any of the maximum values described above. For example, in some embodiments of Formula II, y can be from 10 to 2400 (e.g., from 10 to 1500, from 10 to 1000, from 25 to 1000, or from 25 to 500).

In some embodiments of Formula II, x+y can be at least 5 (e.g., at least 10, at least 15, at least 25, at least 30, at least 40, at least 50, at least 100, at least 200, at least 250, at least 300, at least 400, at least 500, at least 600, at least 700, at least 750, at least 800, at least 900, at least 1000, at least 1100, at least 1200, at least 1250, at least 1300, at least 1400, at least 1500, at least 1600, at least 1700, at least 1750, at least 1800, at least 1900, at least 2000, at least 2100, at least 2200, at least 2300, or at least 2400). In some embodiments of Formula II, x+y can be 2400 or less (e.g., 2300 or less, 2200 or less, 2100 or less, 2000 or less, 1900 or less, 1800 or less, 1750 or less, 1700 or less, 1600 or less, 1500 or less, 1400 or less, 1300 or less, 1250 or less, 1200 or less, 1100 or less, 1000 or less, 900 or less, 800 or less, 750 or less, 700 or less, 600 or less, 500 or less, 400 or less, 300 or less, 250 or less, 200 or less, 100 or less, 50 or less, 40 or less, 30 or less, 25 or less, 15 or less, or 10 or less).

x+y can range from any of the minimum values described above to any of the maximum values described above. For example, in some embodiments of Formula II, x+y can be from 10 to 2400 (e.g., from 10 to 1500, from 10 to 1000, from 25 to 1000, or from 25 to 500).

Also provided herein are methods of treating or preventing a Gram negative bacterial infection in a subject. These methods can comprise administering to the subject a therapeutically or prophylactically effective amount of a polymer-drug conjugate described herein (such as an antibacterial agent-MCP conjugate described herein).

In some embodiments, the Gram negative bacterial infection is caused by a bacteria of one of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella.

Also provided herein are methods of treating or preventing a Gram positive bacterial infection in a subject. These methods can comprise administering to the subject a therapeutically or prophylactically effective amount of a polymer-drug conjugate described herein.

Also provided herein are methods of treating or preventing a bacterial infection in a subject comprising a small colony variant. These methods can comprise administering to the subject a therapeutically or prophylactically effective amount of a polymer-drug conjugate described herein. In some examples, the small colony variant comprises Staphylococcus aureus.

Also provided herein are methods of providing broad spectrum antibiotic therapy to a subject in need thereof to treat or prevent bacterial infection. These methods can comprise administering to the subject a therapeutically or prophylactically effective amount of a polymer-drug conjugate described herein (such as an antibacterial agent-MCP conjugate described herein).

Also provided herein are methods of sensitizing a Gram negative bacteria towards an antimicrobial agent. These methods can comprise contacting the Gram negative bacteria with a polymer-drug conjugate described herein (such as an antibacterial agent-MCP conjugate described herein).

Following sensitization, methods can further comprise contacting the Gram negative bacteria with another antibiotic agent. Examples of suitable antibiotics include antibiotics of the lincomycin family (referring to a class of antibiotic agents originally recovered from Streptomyces lincolnensis); antibiotics of the tetracycline family (referring to a class of antibiotic agents originally recovered from Streptomyces aureofaciens); sulfur-based antibiotics such as the sulfonamides; and so forth. Exemplary antibiotics of the lincomycin family include lincomycin itself (6,8-dideoxy-6-[[(1-methyl-4-propyl-2-pyrrolidinyl)-carbonyl]amino-]-1-thio-L-threo-α-D-galacto-octopyranoside), clindamycin, the 7-deoxy, 7-chloro derivative of lincomycin (e.g., 7-chloro-6,7,8-trideoxy-6-[[(1-methyl-4-propyl-2-pyrrolidinyl) carbonyl]amino]-1-thio-L-threo-α-D-galacto-octopyranoside), and pharmacologically acceptable salts and esters thereof. Exemplary antibiotics of the tetracycline family include tetracycline itself 4-(dimethylamino)-1,4,4α,5,5α,6,11,12α-octahydro-3,6,12,12α-pentahydroxy-6-methyl-1,11-dioxo-2-naphthacenecarboxamide), chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium. Antimicrobials and/or antibiotics further include compounds such as erythromycin, bacitracin, neomycin, penicillin, polymyxin B, tetracyclines, viomycin, chloromycetin and streptomycins, cefazolin, ampicillin, azactam, tobramycin, clindamycin and gentamycin.

In some embodiments, the Gram negative bacterial infection is caused by a bacteria of one of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella.

Also provided herein are medical devices comprising a coating comprising a polymer-drug conjugate described herein.

In some embodiments, the medical device comprises an implantable (e.g., indwelling) medical device. In some embodiments, the medical device is formed at least in part from a metal, a ceramic, a polymer, or a combination thereof.

In some embodiments, the medical device comprises an orthopedic implant or device, a catheter, a suture (resorbable or non-resorbable), a cardiovascular device (e.g., a pacemaker, a stent), an endotracheal tube, a tracheotomy tube, chest tube, a vascular catheter, an urinary catheter, a nephrostomy tube, a biliary stent, a peritoneal catheter, an epidural catheter, a central nervous system catheter, a prosthetic valve, a gastric tube, an intestinal tube, or a drug-delivery implant.

### Click Chemistry and Click Motifs

In some embodiments, the conjugates described above can include a linker comprising a moiety formed by the chemical reaction of two click motifs (i.e., click chemistry).

Click chemistry refers to a class chemical reaction (referred to as a “click reaction”) between two click groups that exhibit good yields, wide functional group tolerance, and are highly selective even in the presence of a complex mixture of biological molecules. These characteristics allow the click reactions to proceed even in vivo. Example click motif pairs used as the first click motif and the second click motif include, but not limited to, azide with phosphine; azide with cyclooctyne; nitrone with cyclooctyne; nitrile oxide with norbornene; oxanorbornadiene with azide; trans-cyclooctene with s-tetrazine; quadricyclane with bis(dithiobenzil)nickel(II).

In some embodiments, the second click motif comprises an alkene, e.g., a cyclooctene, e.g., a transcyclooctene (TCO) or norbornene (NOR), and the first click motif comprises a tetrazine (Tz). In other embodiments, the second click motif comprises an alkyne, e.g., a cyclooctyne such as dibenzocyclooctyne (DBCO), and the first click motif comprises an azide (Az). In some embodiments, the second click motif comprises a Tz, and the first click motif comprises an alkene such as transcyclooctene (TCO) or norbornene (NOR). Alternatively or in addition, the first click motif comprises an Az, and the second click motif comprises a cyclooctyne such as dibenzocyclooctyne (DBCO). TCO reacts specifically in a click chemistry reaction with a tetrazine (Tz) moiety. DBCO reacts specifically in a click chemistry reaction with an azide (Az) moiety. Norbornene reacts specifically in a click chemistry reaction with a tetrazine (Tz) moiety.

Exemplary click chemistry reactions (and by extension click motifs) are shown below. For example, copper(I)-catalyzed Azide-Alkyne Cycloaddition (CuAAC) comprises using a Copper (Cu) catalyst at room temperature. The Azide-Alkyne Cycloaddition is a 1,3-dipolar cycloaddition between an azide and a terminal or internal alkyne to give a 1,2,3-triazole.

Another example of click chemistry includes Staudinger ligation, which is a reaction that is based on the classic Staudinger reaction of azides with triarylphosphines. It launched the field of bioorthogonal chemistry as the first reaction with completely abiotic functional. The azide acts as a soft electrophile that prefers soft nucleophiles such as phosphines. This is in contrast to most biological nucleophiles which are typically hard nucleophiles. The reaction proceeds selectively under water-tolerant conditions to produce a stable product. Phosphines are completely absent from living systems and do not reduce disulfide bonds despite mild reduction potential. Azides had been shown to be biocompatible in FDA-approved drugs such as azidothymidine and through other uses as cross linkers. Additionally, their small size allows them to be easily incorporated into biomolecules through cellular metabolic pathways.

Copper-free click chemistry is a bioorthogonal reaction first developed by Carolyn Bertozzi as an activated variant of an azide alkyne cycloaddition. Unlike CuAAC, Cu-free click chemistry has been modified to be bioorthogonal by eliminating a cytotoxic copper catalyst, allowing reaction to proceed quickly and without live cell toxicity. Instead of copper, the reaction is a strain-promoted alkyne-azide cycloaddition (SPAAC). It was developed as a faster alternative to the Staudinger ligation, with the first generations reacting over sixty times faster. The incredible bioorthogonality of the reaction has allowed the Cu-free click reaction to be applied within cultured cells, live zebrafish, and mice. Cyclooctynes were selected as the smallest stable alkyne ring which increases reactivity through ring strain which has calculated to be 19.9 kcal/mol.

Copper-free click chemistry also includes nitrone dipole cycloaddition. Copper-free click chemistry has been adapted to use nitrones as the 1,3-dipole rather than azides and has been used in the modification of peptides.

This cycloaddition between a nitrone and a cyclooctyne forms N-alkylated isoxazolines. The reaction rate is enhanced by water and is extremely fast with second order rate constants ranging from 12 to 32 M⁻¹ s⁻¹, depending on the substitution of the nitrone. Although the reaction is extremely fast, incorporating the nitrone into biomolecules through metabolic labeling has only been achieved through post-translational peptide modification.

Another example of click chemistry includes norbornene cycloaddition. 1,3 dipolar cycloadditions have been developed as a bioorthogonal reaction using a nitrile oxide as a 1,3-dipole and a norbornene as a dipolarophile. Its primary use has been in labeling DNA and RNA in automated oligonucleotide synthesizers.

Norbornenes were selected as dipolarophiles due to their balance between strain-promoted reactivity and stability. The drawbacks of this reaction include the cross-reactivity of the nitrile oxide due to strong electrophilicity and slow reaction kinetics.

Another example of click chemistry includes oxanorbornadiene cycloaddition. The oxanorbornadiene cycloaddition is a 1,3-dipolar cycloaddition followed by a retro-Diels Alder reaction to generate a triazole-linked conjugate with the elimination of a furan molecule. This reaction is useful in peptide labeling experiments, and it has also been used in the generation of SPECT imaging compounds.

Ring strain and electron deficiency in the oxanorbornadiene increase reactivity towards the cycloaddition rate-limiting step. The retro-Diels Alder reaction occurs quickly afterwards to form the stable 1,2,3 triazole. Limitations of this reaction include poor tolerance for substituents which may change electronics of the oxanorbornadiene and low rates (second order rate constants on the order of 10⁻⁴).

Another example of click chemistry includes tetrazine ligation. The tetrazine ligation is the reaction of a trans-cyclooctene and an s-tetrazine in an inverse-demand Diels Alder reaction followed by a retro-Diels Alder reaction to eliminate nitrogen gas. The reaction is extremely rapid with a second order rate constant of 2000 M⁻¹-s⁻¹(in 9:1 methanol/water) allowing modifications of biomolecules at extremely low concentrations.

The highly strained trans-cyclooctene is used as a reactive dienophile. The diene is a 3,6-diaryl-s-tetrazine which has been substituted in order to resist immediate reaction with water. The reaction proceeds through an initial cycloaddition followed by a reverse Diels Alder to eliminate N₂ and prevent reversibility of the reaction.

Not only is the reaction tolerant of water, but it has been found that the rate increases in aqueous media. Reactions have also been performed using norbornenes as dienophiles at second order rates on the order of 1 M⁻¹·s⁻¹ in aqueous media. The reaction has been applied in labeling live cells and polymer coupling.

Another example of click chemistry includes is [4+1]cycloaddition. This isocyanide click reaction is a [4+1]cycloaddition followed by a retro-Diels Alder elimination of N₂.

The reaction proceeds with an initial [4+1]cycloaddition followed by a reversion to eliminate a thermodynamic sink and prevent reversibility. This product is stable if a tertiary amine or isocyanopropanoate is used. If a secondary or primary isocyanide is used, the produce will form an imine which is quickly hydrolyzed.

Isocyanide is a favored chemical reporter due to its small size, stability, non-toxicity, and absence in mammalian systems. However, the reaction is slow, with second order rate constants on the order of 10⁻²M⁻¹ s⁻¹.

Another example of click chemistry includes quadricyclane ligation. The quadricyclane ligation utilizes a highly strained quadricyclane to undergo [2+2+2]cycloaddition with π systems.

Quadricyclane is abiotic, unreactive with biomolecules (due to complete saturation), relatively small, and highly strained (~80 kcal/mol). However, it is highly stable at room temperature and in aqueous conditions at physiological pH. It is selectively able to react with electron-poor π systems but not simple alkenes, alkynes, or cyclooctynes.

Bis(dithiobenzil)nickel(II) was chosen as a reaction partner out of a candidate screen based on reactivity. To prevent light-induced reversion to norbornadiene, diethyldithiocarbamate is added to chelate the nickel in the product.

These reactions are enhanced by aqueous conditions with a second order rate constant of 0.25 M⁻¹ s⁻¹. Of particular interest is that it has been proven to be bioorthogonal to both oxime formation and copper-free click chemistry.

The exemplary click chemistry reactions have high specificity, efficient kinetics, and occur in vivo under physiological conditions. See, e.g., Baskin et al. Proc. Natl. Acad. Sci. USA 104(2007):16793; Oneto et al. Acta biomaterilia (2014); Neves et al. Bioconjugate chemistry 24(2013):934; Koo et al. Angewandte Chemie 51(2012):11836; and Rossin et al. Angewandte Chemie 49(2010):3375. For a review of a wide variety of click chemistry reactions and their methodologies, see e.g., Nwe K and Brechbiel M W, 2009 Cancer Biotherapy and Radiopharmaceuticals, 24(3): 289-302; Kolb H C et al., 2001 Angew. Chem. Int. Ed. 40: 2004-2021. The entire contents of each of the foregoing references are incorporated herein by reference.

Exemplary click motif pairs are shown in the table below. Functional groups formed by reaction of click motif pairs are well known in the art.

| Functional | | | |
|---|---|---|---|
| group/Click | Paired | | Reaction type |
| Motif | with | Functional group/Click Motif | (Reference) |
| azide | | Phosphine | Staudinger ligation |
| | | | (Saxon et al. Science |
| | | | 287(2000):2007-10) |
| azide | | Cyclooctyne, e.g., dibenzocyclooctyne, one of | Copper-free click |
| | | the cyclooctynes shown below, or other similar | chemistry (Jewett et al. J. |
| | | cyclooctynes: | Am. Chem. Soc. |
| | | 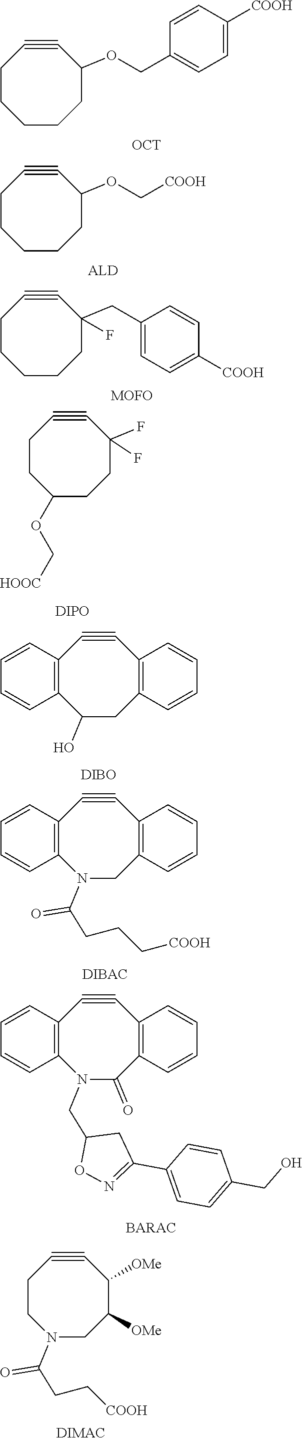 | 132.11(2010):3688-90; Sletten et al. Organic Letters 10.14 (2008):3097-9; Lutz. Angew. Chem., Int. Ed 47.12(2008):2182) |
| nitrone | | Cyclooctyne | Nitrone Dipole |
| | | | Cycloaddition (Ning et |
| | | | al. Angew. Chem., Int. Ed |
| | | | 49.17 (2010):3065) |
| Nitrile oxide | | Norbornene | Norbornene |
| | | | Cycloaddition |
| | | | (Gutsmiedl et al. Organic |
| | | | Letters |
| | | | 11.11(2009):2405-8) |
| oxanorbornadiene | | Azide | Oxanorbornadiene |
| | | | Cycloaddition (Van |
| | | | Berkel et al. |
| | | | ChemBioChem |
| | | | 8.13(2007):1504-8) |
| Trans-cyclooctene, | | s-tetrazine | Tetrazine ligation |
| norbornene, or | | | (Hansell et al. J. Am. |
| other alkene | | | Chem. Soc. |
| | | | 133.35(2011):13828-31) |
| nitrile | | 1,2,4,5-tetrazine | [4 + 1] cycloaddition |
| | | | (Stackman et al. Organic |
| | | | and Biomol. Chem. |
| | | | 9.21(2011):7303) |
| quadricyclane | | Bis(dithiobenzil)nickel(II) | Quadricyclane Ligation |
| | | | (Sletten et al. J. Am. |
| | | | Chem. Soc. |
| | | | 133.44(2011): 17570-3) |
| Ketone or aldehyde | | Hydrazines, hydrazones, oximes, amines, ureas, | Non-aldol carbonyl |
| | | thioureas, etc. | chemistry |
| | | | (Khomyakova EA, et al. |
| | | | Nucleosides Nucleotides |
| | | | Nucleic Acids. 30(7-8) |
| | | | (2011) 577-84 |
| Thiol | | Maleimide | Michael addition |
| | | | (Zhou et al. Bioconjug |
| | | | Chem 2007 18(2):323- |
| | | | 32.) |
| Dienes | | Dienophiles | Diels Alder (Rossin et al. |
| | | | Nucl Med. (2013) |
| | | | 54(11):1989-95) |
| Tetrazine | | norbornene, propene, trans-cyclooctene, other | |
| | | strained alkenes. | |

Other suitable include the motifs can be found, for example, in Patterson, D. M., et al. “Finding the Right (Bioorthogonal) Chemistry,” ACS Chem. Biol., 2014, 9(3): 592-605; Akgun, B., et al. “Synergic “Click” Boronate/Thiosemicarbazone System for Fast and Irreversible Bioorthogonal Conjugation in Live Cells,” J Am. Chem. Soc., 2017, 139(40): 14285-14291; and Akgun, B. and Hall, D. G. “Fast and Tight Boronate Formation for Click Bioorthogonal Conjugation,” Angew. Chem., Int. Ed. 2016, 55(12): 3909-3913, each of which is hereby incorporated by reference in its entirety.

### Medical Devices

A “medical device” is defined herein to refer to an instrument, apparatus, implement, machine, contrivance, implant, or other similar or related article, including a component part, or accessory which is intended for use in the diagnosis, treatment, or prevention of disease or other health-related conditions in a subject. The subject can be any vertebrate, such as a mammal. In particular embodiments, the subject is a human. Non-limiting examples of medical devices include a stent, a tube, a catheter, or a valve.

A “stent” as used herein refers to a thread, rod, or catheter inserted into a tubular structure, such as a blood vessel, to provide support during or after anastomosis.

A “catheter” is defined herein to refer to a hollow tube (which may or may not be flexible) for insertion into a body cavity, duct, or vessel to allow the passage of fluids or distend a passageway.

Particular non-limiting examples of medical devices include an endotracheal tube, tracheotomy tube, ureteral stent, biliary stent, ventriculostomy catheters, chest tube, a vascular catheter, an urinary catheter, a gastric tube, an intestinal tube, a nephrostomy tube, a biliary stent, a peritoneal catheter, an epidural catheter, a central nervous system catheter, an orthopedic device, a prosthetic valve, orthopedic devices, pacemakers, infusion pumps, infusion ports, dialysis catheters, neurotransmitters, drug-delivery implant, and gloves. Non-limiting examples of vascular catheters include of a central venous catheter, an arterial line, a pulmonary artery catheter, a stent, and a peripheral venous catheter.

Other medical devices that can benefit from the present invention include blood exchanging devices, vascular access ports, cardiovascular catheters, extracorpeal circuits, stents, implantable prostheses, vascular grafts, pumps, heart valves, and cardiovascular sutures, to name a few. Regardless of detailed embodiments, applicability of the invention should not be considered limited with respect to the type of medical device, implant location or materials of construction of the device.

Other examples of non-organic surfaces contemplated by the present invention include surfaces of medical supplies and medical equipment. Non-limiting examples of medical supplies and equipment include gloves (such as disposable gloves), gowns, pads, wheelchairs, stretchers, tables, swabs, sponges, sutures (such as silk sutures), bags, surgical supplies, and packaging materials for the packaging of sterile medical or hospital supplies.

Further examples of non-organic surfaces include the surfaces of a sponge, wipe, pad, or mop.

Further examples of non-organic surfaces include floors, countertops, the surface of a container (such as for food storage), and the surfaces of food processing supplies and equipment, mopping equipment or sponges. Still further examples of non-organic surfaces include the surface of personal care products such as gloves, condoms, diaphragms, and sanitary napkins.

The present application will now be described in connection with certain embodiments, which are not intended to limit the scope of the invention. On the contrary, the present application covers all alternatives, modifications, and equivalents as included within the scope of the claims. Thus, the following will illustrate the practice of the present application, for the purposes of illustration of certain embodiments and is presented to provide what is believed to be a useful and readily understood description of its procedures and conceptual aspects.

### Examples

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

Synthesis and Characterization of Vancomycin-Modified Hyaluronic Acids Materials. Hyaluronic acid sodium salt (HA, 100 kD & 10-25 kD, medical grade, Echelon Biosciences) was used without further purification. Polyacrylic acid (PAA, 50 kD, 25 wt % in water, Polysciences) was neutralized to pH 6.5 with 1N sodium hydroxide aq. solution before being subjected to chemical conjugation with antibiotics. Coupling reagents, 1-ethyl-3-(3-dimethylaminopropyl) carbodiimide hydrochloride (EDC) and N-hydroxysuccinimide (NHS, 98%) were purchased from TCI and Thermo Scientific, respectively. 4-Morpholineethanesulfonic acid (MES) were purchased from Sigma-Aldrich. Vancomycin hydrochloride was purchased from Slate Run Pharmaceutics. Bioluminescent Staphylococcus aureus Xen29 (S. aureus) was purchased from Perkin Elmer. Escherichia coli ATCC™43888™ (E. coli) was purchased from Fisher Scientific. Fisher brand Luria-Bertani (LB) broth and LB agar were used.

Preparation of HA-Vancomycin (HA-Vanco) and PAA-Vancomycin (PAA-Vanco) conjugates. HA or PAA (100 mg, 1 eq.) was first dissolved in MES buffer (pH 6.5), to which EDC (2 eq.) and NHS (1 eq.) were added sequentially in 5 min each under stirring (300 rpm). Vancomycin (0.5 eq. for HA; 0.04-0.5 eq. for PAA) was dissolved in MES buffer and slowly added to the HA/EDC/NHS mixture, resulting in a ratio of amine to carboxyl group (R) to be 0.5 for HA and 0.04-0.5 for PAA. The mixture (15-25 mL) was stirred for 24 h at 300 rpm. The mixture was then transferred to a centrifuge tube. Sodium chloride (1.7 g/10 mL of mixture) was added and fully dissolved in the mixture to screen the charges of HA chains. HA-antibiotic conjugates were precipitated by ethanol (up to 67% v/v), pelleted by centrifugation (2000 rpm, 5 min), redissolved in 10 mL of MilliQ water, and dialyzed (MWCO, 10 kD) against 1 L of MilliQ water for 3 days with water changes twice a day. The purified HA-Vanco and PAA-Vanco were lyophilized and stored in a desiccator at room temperature (RT).

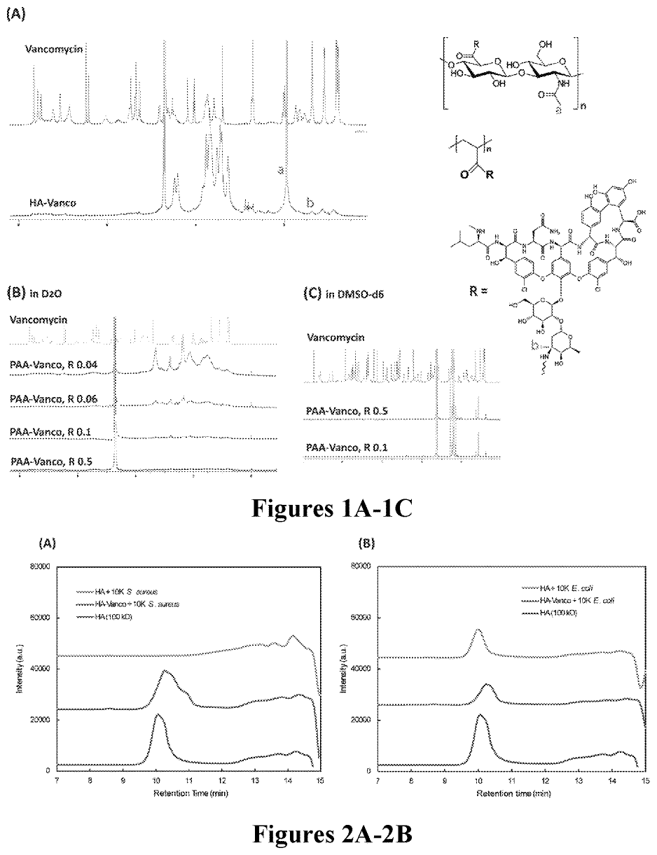

NMR of HA-Vanco and PAA-Vanco Conjugates. Degree of substitution (DS) of the HA-Vanco was determined by ¹H NMR (Bruker Plus 400 MHz spectrometer) in D₂O. DS (mol %) is defined as the average number of substituent groups per disaccharide repeating unit of HA, as calculated by the ratio of integrations of the characteristic protons of vancomycin (b in FIG. 1A) to that of the methyl group of the HA repeating unit (a in FIG. 1A). Average DS (%) of HA-Vanco is 6.5±0.3.

The high coupling efficiency of PAA using EDC/NHS was observed. PAA-Vanco rapidly self-assembled in aq. solution and turned turbid upon vancomycin conjugation (R>0.04), possibly driven by the non-covalent interactions among the conjugated vancomycin. As a result, the DS of PAA-Vanco cannot be readily obtained from ¹H NMR due to insolubility of the self-assemblies (R>0.04) in water and the overlapping signals between PAA backbone and vancomycin in D₂O (R=0.04) (FIG. 1). Instead, we confirmed that no vancomycin was left unreacted by extracting the freeze-dried PAA-Vanco with DMSO-d6 for NMR validation (PAA-Vanco conjugate was insoluble in DMSO while vancomycin was soluble in DMSO) (FIG. 1C).

Determination of the degree of cleavage of HA and HA-Vanco conjugates upon in vitro incubation with S. aureus or E. coli. HA is subject to cleavage by bacterial hyaluronidase. HA, or HA-Vanco were separately dissolved in PBS (pH 7.4, 5 mg/mL), to which the S. aureus or E. coli suspension in LB (10,000 CFU) was added. The mixture was diluted with LB to obtain the final volume ratio of PBS/LB=1:1, and incubated at 37° C. overnight. HA and HA-Vanco solutions were also incubated at 37° C. overnight in the absence of any bacteria as controls. After the overnight incubation, the samples were centrifuged at 3000 rpm for 5 min to pellet the bacteria and collect supernatant. Enzyme activity of any secreted bacterial enzymes was halted by heating the supernatant in boiling water bath for 10 min. Upon cooling, the supernatant was subjected to GPC analysis to determine the degree of HA or HA-Vanco conjugate cleavage by bacterial enzyme during the incubation.

The molecular weight of HA or HA-Vanco with and without prior incubation with bacterial suspension cultures were determined by GPC equipped with two AqualGel 20-OH columns (Agilent) and a reflective index detector. Samples were first diluted with MilliQ water to 2.5 mg/mL and filtered with 0.45-μm syringe filters. Elution was carried out for 30 min at a flow rate of 1 mL/min with 0.2 M of sodium nitrate and 0.01 M of monobasic sodium phosphate (pH 7.0) as eluent.

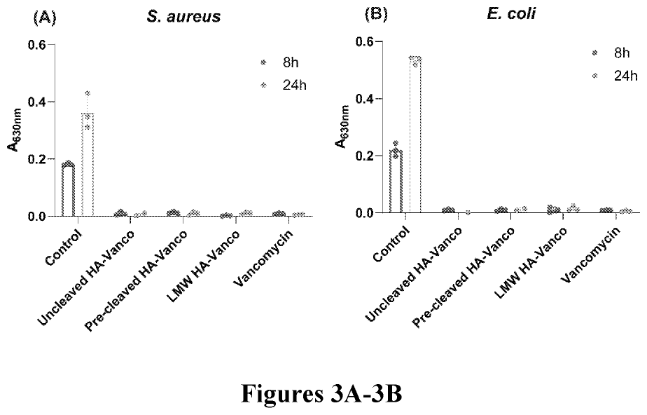

As shown in FIG. 2A, HA was fully cleaved by extracellularly secreted S. aureus hyaluronidase upon overnight incubation with S. aureus. By contrast, most of HA-Vanco in the solution remained intact, suggesting that S. aureus may have been rapidly killed by HA-Vanco before they were able to secret adequate hyaluronidase to degrade the HA-Vanco. FIG. 2B showed that both HA and HA-Vanco in the solution remained intact upon overnight incubation with E. coli, consistent with the lack of extracellularly secreted hyaluronidase by E. coli.

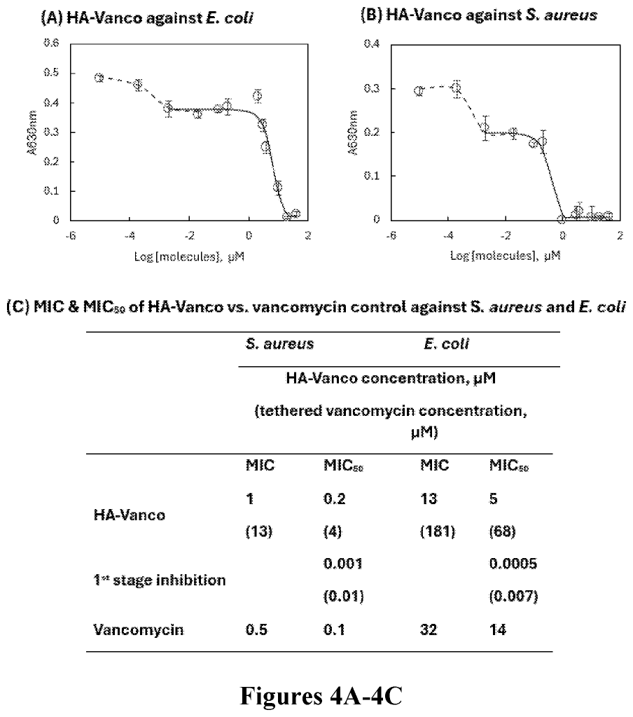

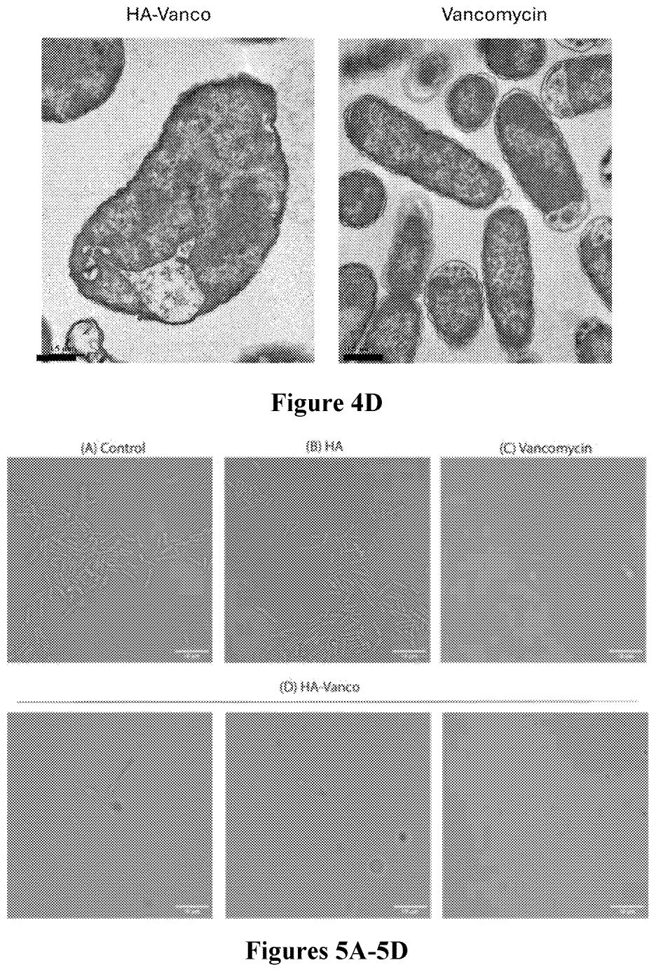

Antibacterial properties of HA-Vanco conjugate, those pre-cleaved by bacterial hyaluronate lyase, low-MW HA-Vanco conjugate vs. unmodified vancomycin against S. aureus and E. coli. High-MW HA-Vanco were dissolved in PBS (1 mL) with a fixed content of the vancomycin conjugated (500 μM). Bacterial hyaluronate lyase (10 U) was added to the solutions and incubated overnight at 37° C. to obtain pre-cleaved samples. HA and HA-Vanco conjugates were also incubated without enzyme (i.e. uncleaved) at 37° C. overnight as intact controls. The antibacterial activities of pre-cleaved and uncleaved HA-Vanco, along with lower MW HA-Vanco and unmodified vancomycin, were determined by incubating respective solutions with S. aureus or E. coli for 24 h on a 96-well plate. Each well contained intact or pre-cleaved HA-Vanco conjugates or unmodified vancomycin PBS solution (100 μL) and 10,000-CFU S. aureus or E. coli in LB broth. LB broth was added to bring the total volume of each well to 200 μL. The wells containing PBS (100 μL) and 10,000-CFU S. aureus or E. coli in LB (100 μL) serve as no-treatment control. Absorbance was measured at time 0, 8 and 24 h at 630 nm (A₆₃₀ₙₘ) on a plate reader (MSLER07, NEUVAR INC.) and the absorbance at 0 h was used for background subtraction. As shown by FIG. 3, all HA-Vanco conjugates, regardless of MW or pre-cleavage by hyaluronidase, were able to effectively kill S. aureus and E. coli in suspension culture.

Determination of minimum inhibitory concentration (MIC) of HA-Vanco conjugates against S. aureus and E. coli. HA-Vanco and unmodified vancomycin were dissolved in PBS, serially diluted and added to a 96-well plate for bacterial suspension culture. Each well contained 100 μL of HA-Vanco conjugate or unmodified vancomycin of a given concentration in PBS and 10,000-CFU S. aureus or E. coli in LB broth. The final volume in each well was brought to 200 μL by additional LB to achieve the volume ratio of PBS/LB=1:1. The plate was incubated at 37° C. for 24 h. A₆₃₀ was then measured on a plate reader. The logarithm of HA-Vanco concentration was plotted against the absorbance. A curve fitting using a modified Gompertz function was performed by the least squares method. The MIC was derived from the best fit curve parameters as defined by Lambert and Pearson (2000). MIC₅₀ was defined as the HA-Vanco conjugate or vancomycin concentration at which A₆₃₀ is halfway between minimum and maximum absorbance defined by the best fit curve. MIC is presented with units both in the concentration of conjugated vancomycin in HA-Vanco (given by monomer concentration×DS), and the concentration of HA-Vanco molecules (given by monomer concentration/degree of polymerization of starting HA).

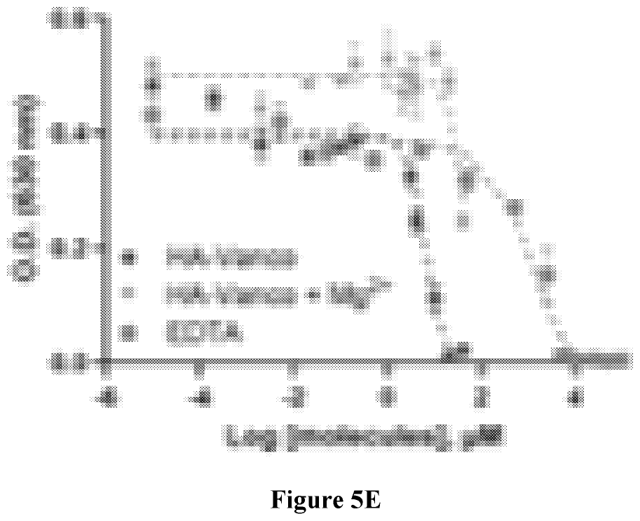

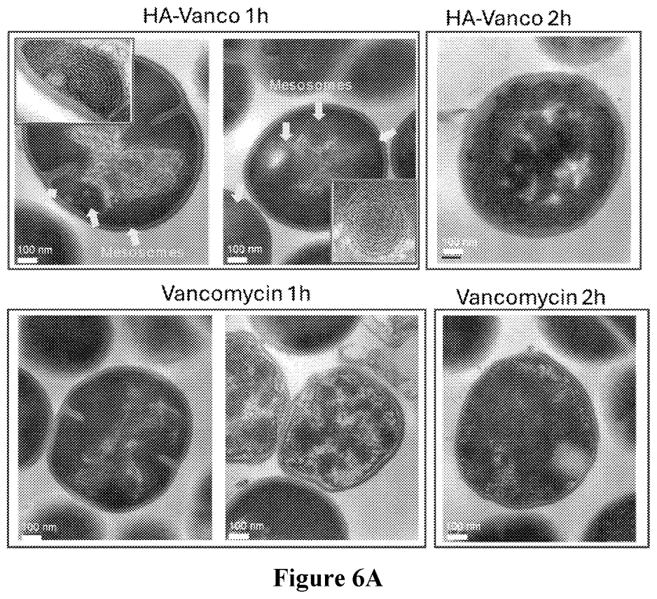

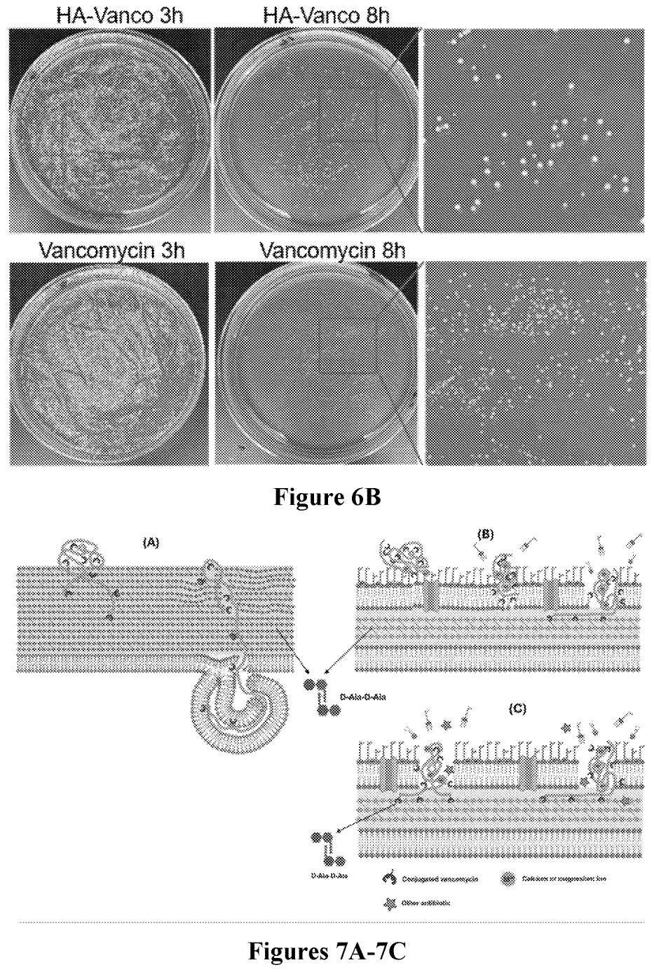

Morphological changes of bacteria treated with HA-Vanco conjugate vs. vancomycin. HA, or HA-Vanco were dissolved in PBS with fixed molecular concentration of 100 μM. The HA, HA-Vanco, along with unmodified vancomycin were incubated with E. coli for 3 h in microcentrifuge tubes before being collected for microscopy. Each tube contained HA, HA-Vanco conjugates or unmodified vancomycin PBS solutions (300 μL) and 300,000-CFU E. coli in LB broth. LB broth was added to bring the volume of each well to 600 μL. The wells containing PBS (300 μL) and 300,000-CFUE. coli in LB (300 μL) serve as no-treatment control. E. coli was centrifuged down (18,000×g, 3 min) and the pellet was washed with PBS once before fixation with 10% formalin for 20 min at RT. Fixed E. coli was pelleted and washed with PBS once and re-dispersed in 50 μL of PBS and placed onto poly-lysine coated coverslips. After DAPI staining, PBS wash, and mounting with an aqueous mounting medium, the fixed cells were observed by a Leica TCS SP5 II confocal laser scanning microscope (CLSM). Overlay of DAPi staining and bright field images shown in FIGS. 5A and 5B revealed normal morphology of E. coli with HA treatment, validating that HA has no impact on E. coli morphology or viability. As shown in FIG. 5C, after 3 h treatment with vancomycin, some E. coli could still be observed on the slide, with near normal cell morphology, supporting that the killing by vancomycin was not super-effective for the Gram negative bacteria. By contrast, with HA-Vanco treatment, no live E. coli with normal cell morphology was observed, with very few detected on the slide showing cell blebbing (FIGS. 5 D-F), an outcome commonly observed in Gram negative bacteria with significantly disrupted cell wall.

To investigate the detailed mechanism of action of HA-Vanco on E. coli and S. aureus, PBS, HA, HA-Vanco or Vanco solutions (50 μM) was co-cultured with 300,000-CFU E. coli or 6,000,000-CFU S. aureus in PBS/LB 1:1 media in microcentrifuge tubes for 3 h (E. coli) and 1-2 h (S. aureus) at 37° C. Bacteria was centrifuged down (2900×g, 5 min) and the pellet was washed with cold PBS once before fixation with a fixative (2.5% glutaraldehyde/1.6% paraformaldehyde in 1M sodium cacodylate buffer) overnight. Bacteria was then briefly rinsed (10 mins) in 1 M sodium cacodylate buffer (pH 7.2) thrice and post-fixed for 1 h in 1% osmium tetroxide/H₂O (w/v). The samples were then finally washed (10 mins) with water thrice and left overnight. The next step, bacteria pellets were dehydrated through a graded series of ethanol of 10%, 30%, 50%, 70% (10 min/each), then 85%, 95% (20 min/each) and finally 100% ethanol for 20 min thrice. Samples were then infiltrated with two changes of 100% propylene oxide before being placed in a 50%/50% propylene oxide/SPI-Pon 812 resin mixture overnight. Then, bacteria pellets were transferred through four changes of fresh SPI-Pon 812 epoxy resin and finally embedded in tubes filled with the same resin and polymerized for 48 h at 70° C. The epoxy blocks were then trimmed, and ultrathin sections (70 nm) were cut on a Leica EM UC7 ultramicrotome using a Diatome diamond knife. The sections were collected and mounted on copper support grids and contrasted with lead citrate and uranyl acetate. The samples were examined on a Philips CM10 Electron Microscope using 100 KV accelerating voltage. Images were captured using a Gatan Erlangshen ES1000W 785 TEM CCD camera.

Antibacterial properties of PAA-Vanco conjugate against S. aureus and E. coli. Following the antibacterial properties measurement forementioned, PAA-Vanco with R from 0.04 to 0.5 were dissolved in PBS (1 mL) with either a fixed solid concentration (8.3 mg/mL, comparable with the one HA-Vanco in FIG. 3), or a fixed content of the vancomycin conjugated (500 μM), assuming 100% coupling efficiency. PAA-Vanco with R=0.06, 0.1 and 0.5 were subject to probe sonication (10 s×3; VCX130A, SONICS) in an ice bath to disperse PAA-Vanco in PBS. The antibacterial activities of PAA-Vanco were determined by co-incubating with S. aureus or E. coli for 24 h in a 96-well plate. Each well contained PAA-Vanco conjugates with a given R in PBS solution (100 μL) and 10,000-CFU S. aureus or E. coli in LB broth. LB broth was added to bring the total volume of each well to 200 μL. The wells containing PBS (100 μL) and 10,000-CFU S. aureus or E. coli in LB (100 μL) serve as no-treatment control. To confirm clearance of bacteria, 10 μL of mixture was taken and spread on an agar plate for culture, and detection of any live bacterial colony, even if CFU counts significantly lower than that of the untreated control, would be interpreted as “partially inhibited” as shown in Table 1

TABLE 1
Antibacterial properties of LMW HA-Vanco and PAA-Vanco with
R from 0.5 to 0.04 at either 4.2 mg/mL (comparable concentration
with HA-Vanco in FIG. 3) or at fixed antibiotic concentration
of 250 μM assuming 100% coupling efficiency.

| | Concentration | 10,000 CFU | 10,000 CFU |
|---|---|---|---|
| | (mg/mL) | S. aureus** | E. coli |
| LMW HA-Vanco | 4.2 | Clearance | Clearance |
| PAA-Vanco R | 4.2 | Clearance | Clearance |
| 0.5 | 0.8* | Clearance | Clearance |
| PAA-Vanco R | 4.2 | Clearance | Clearance |
| 0.1 | 1.1* | Clearance | Partially inhibited |

*The concentration made to have fixed antibiotic concentration of 250 μM assuming 100% coupling efficiency.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method of improving the activity of an antibacterial agent against a Gram-negative bacteria, the method comprising conjugating the antibacterial agent to a metal-chelating polymer (MCP).

2. The method of claim 1, wherein the MCP has a molecular weight of from 4000 Da to 500 kDa, such as a molecular weight of from 50 kDa to 100 kDa.

3. The method of any one of claims 1-2, wherein the MCP comprises a synthetic polymer.

4. The method of any one of claims 1-2, wherein the MCP comprises a biopolymer.

5. The method of claim 4, wherein the MCP comprises a polysaccharide.

6. The method of any one of claims 1-5, wherein the MCP comprises a polymer bearing multiple carboxylic acid moieties, such as a poly(meth)acrylic acid or a copolymer thereof (e.g., poly(acrylic acid-co-maleic acid) (PAM)), a polyamine polymer, such as polyethyleneimine (PEI) or a copolymer thereof, a polymer bearing multiple iminodiacetic acid (IDA) moieties, such as a polymer derived from glycidyl methacrylate (GMA) and then modified to include modified to include IDA moieties, or a polysaccharide, such as chitosan, alginate, pectin, cellulose, glycogen, chitin, starch such as potato starch, glycosaminoglycans such as chondroitin, chondroitin sulfate, dermatan sulfate, hyaluronic acid, and keratan sulfate; aminated dextrans including DEAE-dextran; aminated starch, aminated glycogen, aminated cellulose, aminated pectin, and salts, complexes, derivatives and mixtures thereof.

7. The method of any one of claims 1-6, wherein the antibacterial agent is covalently bound to the MCP via a non-cleavable linker.

8. The method of any one of claims 1-6, wherein the antibacterial agent is covalently bound to the MCP via a cleavable linker.

9. The method of any one of claims 1-8, wherein the MCP is a negatively charged polymer.

10. The method of any one of claims 1-9, wherein the antibacterial agent is an antibiotic that is indicated for use against a Gram positive bacterial infection.

11. The method of any one of claims 1-10, wherein the antibacterial agent is an antibiotic that is not indicated for use against a Gram negative bacterial infection.

12. The method of any one of claims 1-11, wherein the antibacterial agent comprises a glycopeptide antibiotic, such as vancomycin, teicoplanin, telavancin, oritavancin, dalbavancin, bleomycin, ramoplanin, or decaplanin.

13. The method of any one of claims 1-12, wherein the antibacterial agent comprises a large beta-lactam antibiotic (i.e., a beta-lactam antibiotic that is not indicated for use against a Gram negative bacterial infection), such as penicillin G or nafcillin/oxacillin.

14. The method of any one of claims 1-13, wherein the antibacterial agent comprises a macrolide antibiotic, such as erythromycin, clarithromycin, azithromycin, roxithromycin, telithromycin, fidaxomicin, oleandomycin, methymycin, or tylosin.

15. The method of any one of claims 1-14, wherein the antibacterial agent comprises a lipopeptide antibiotic, such as daptomycin, polymyxins (colistin and polymyxin B), surfactin, iturin, or fengycin; or a lincosamide antibiotic, such as clindamycin.

16. The method of any one of claims 1-15, further comprising conjugating a second antibacterial agent to the MCP.

17. The method of claim 16, wherein the second antibacterial agent comprises an antibiotic that is not indicated for use against both a Gram negative bacterial infection and a Gram positive bacterial infection (e.g., a broad spectrum antibiotic), and wherein the second antibacterial agent is covalently bound to the MCP optionally via a cleavable linker.

18. The method of any one of claims 16-17, wherein the second antibacterial agent comprises doxycycline, minocycline, an aminoglycoside antibiotic (other than streptomycin), ampicillin, amoxicillin/clavulanic acid (Augmentin), azithromycin, a carbapenem antibiotic (e.g., imipenem), piperacillin/tazobactam, a quinolone antibiotic (e.g., ciprofloxacin), a tetracycline antibiotic (other than sarecycline), chloramphenicol, ticarcillin, trimethoprim/sulfamethoxazole (Bactrim), ofloxacin, or a combination thereof.

19. The method of any one of claims 1-18, wherein conjugating the antibacterial agent to the MCP affords an antibacterial agent-MCP conjugate comprising one or more covalently modified monomers defined by Formula I 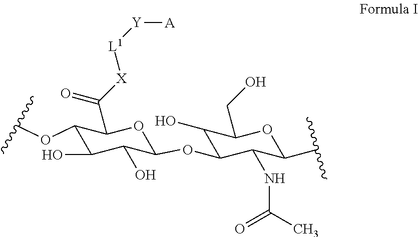
wherein
A is an antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

20. The method of claim 19, wherein the linking group comprises a moiety formed by the chemical reaction of two click motifs, and wherein conjugating the antibacterial agent to the MCP comprises performing a click reaction between an MCP bearing a first click motif and an antibacterial agent bearing a second click motif.

21. The method of any one of claims 1-20, wherein conjugating the antibacterial agent to the MCP affords an antibacterial agent-MCP conjugate comprising a random copolymer defined by Formula II below 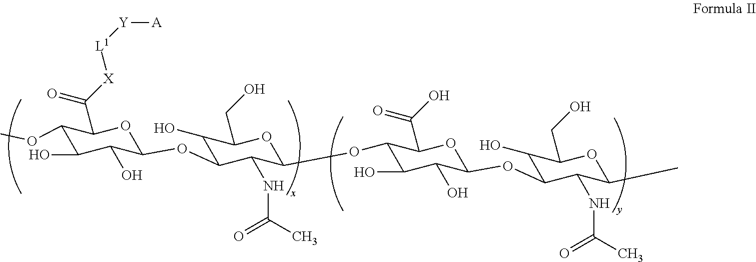
wherein
x and y are each independently integers from 1 to 2500, wherein x+y is no more than 2500, and wherein x and y represent the relative portion of each monomer within the random copolymer;
A is an antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

22. The method of any one of claims 1-21, wherein conjugating the antibacterial agent to the MCP affords an antibacterial agent-MCP conjugate comprising a random copolymer defined by Formula III below 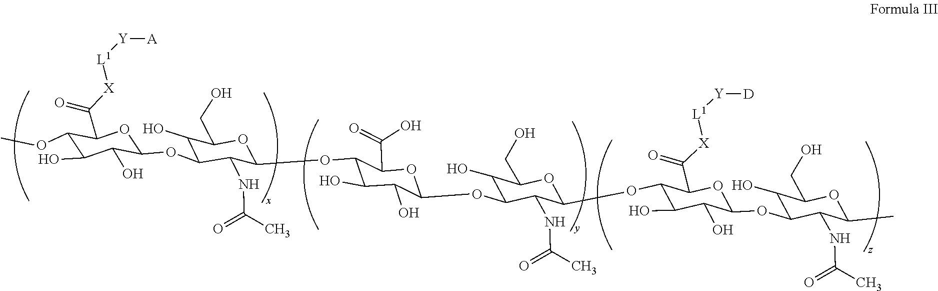
wherein
x, y, and z are each independently integers from 1 to 2500, wherein x+y+z is no more than 2500, and wherein x, y, and z represent the relative portion of each monomer within the random copolymer;
A is a first antibacterial agent;
D is a second antibacterial agent;
Y is absent, or is chosen from —O(C═O)—, —C(═O)—, —S(O)ₓ—, —C(═O)R^{a}C(═O)—, —NR^{a}C(═O)—, —NC(═O)R^{a}C(═O)—, or —C(═O)NR^{a}C(═O)—;
L¹ is absent, or is a linking group; and
X is absent, or is chosen from NH, O, or S.

23. The method of claim 22, wherein the first antibacterial agent comprises an antibacterial agent that targets bacterial cell walls, and the second antibacterial agent comprises an antibacterial agent that targets other structural components such as the cytoplasmic membrane of bacterial cells.

24. The method of claim 23, wherein the antibacterial agent that targets bacterial cell walls is chosen from a β-lactam antibiotic, a glycopeptide antibiotic, a lipoglycopeptide antibiotic, and bacitracin, optionally wherein the antibacterial agent that targets bacterial cell walls is a penicillin, a cephalosporin, a carbapenem, vancomycin, teicoplanin, dalbavancin, oritavancin, telavancin, or a pharmaceutically acceptable salt thereof.

25. The method of any one of claims 23-24, wherein the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

26. A polymer-drug conjugate comprising an antibacterial agent conjugated to a polymer,
wherein the polymer-drug conjugate exhibits a molecular weight of at least 5,000 Da when active intracellularly within a target bacterial cell.

27. The conjugate of claim 26, wherein the polymer-drug conjugate interacts with at least two different structural components of the target bacterial cell to produce cytotoxic activity.

28. The conjugate of claim 27, wherein the target bacterial cell comprises a Gram positive bacteria, and the polymer-drug conjugate interacts with a cell wall of the Gram positive bacteria and a cytoplasmic membrane of the Gram positive bacteria.

29. The conjugate of any one of claims 27-28, wherein the polymer-drug conjugate is amphiphilic.

30. The conjugate of any one of claims 27-29, wherein the polymer-drug conjugate comprises a hydrophobic antibacterial agent conjugated to a hydrophilic polymer.

31. The conjugate of any one of claims 27-30, wherein the hydrophobic antibacterial agent comprises an antibacterial agent that targets bacterial cell walls.

32. The conjugate of claim 31, wherein the hydrophobic antibacterial agent is chosen from a β-lactam antibiotic, a glycopeptide antibiotic, a lipoglycopeptide antibiotic, and bacitracin, optionally wherein the antibacterial agent that targets bacterial cell walls is a penicillin, a cephalosporin, a carbapenem, vancomycin, teicoplanin, dalbavancin, oritavancin, telavancin, or any combination thereof.

33. The conjugate of any one of claims 27-32, wherein the polymer-drug conjugate further comprises a second antibacterial agent conjugated to the polymer, wherein the second antibacterial agent comprises an agent that targets the cytoplasmic membrane of bacterial cells.

34. The conjugate of claim 33, wherein the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

35. The conjugate of any one of claims 30-34, wherein the hydrophilic polymer comprises a polysaccharide, such as hyaluronic acid.

36. The conjugate of any one of claims 28-35, wherein the polymer-drug conjugate comprises one or more covalently modified monomers defined by Formula I 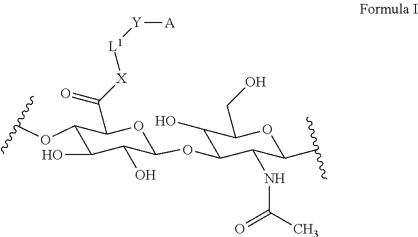
wherein
A is an antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

37. The conjugate of claim 36, wherein the linking group comprises a moiety formed by the chemical reaction of two click motifs.

38. The conjugate of any one of claims 28-37, wherein the polymer-drug conjugate comprises a random copolymer defined by Formula II below 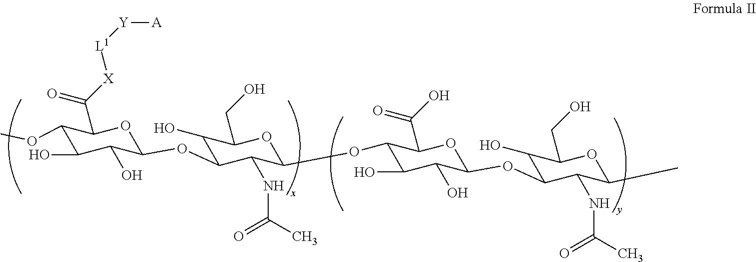
wherein
x and y are each independently integers from 1 to 2500, wherein x+y is no more than 2500, and wherein x and y represent the relative portion of each monomer within the random copolymer;
A is an antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

39. The conjugate of any one of claims 28-38, wherein the polymer-drug conjugate comprises a random copolymer defined by Formula III below 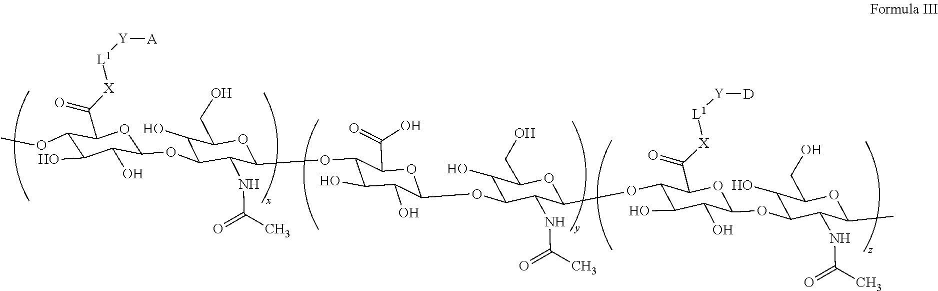
wherein
x, y, and z are each independently integers from 1 to 2500, wherein x+y+z is no more than 2500, and wherein x, y, and z represent the relative portion of each monomer within the random copolymer;
A is a first antibacterial agent;
D is a second antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

40. The conjugate of claim 39, wherein the first antibacterial agent comprises an antibacterial agent that targets bacterial cell walls, and the second antibacterial agent comprises an antibacterial agent that targets other structural components such as the cytoplasmic membrane of bacterial cells.

41. The conjugate of claim 40, wherein the antibacterial agent that targets bacterial cell walls is chosen from a β-lactam antibiotic, a glycopeptide antibiotic, a lipoglycopeptide antibiotic, and bacitracin, optionally wherein the antibacterial agent that targets bacterial cell walls is a penicillin, a cephalosporin, a carbapenem, vancomycin, teicoplanin, dalbavancin, oritavancin, telavancin, or a pharmaceutically acceptable salt thereof.

42. The conjugate of any one of claims 40-41, wherein the agent that targets the cytoplasmic membrane of bacterial cells is chosen from a cationic antimicrobial peptide, a lipopeptide antibiotic, a polymyxin, a carbazole-based small molecule, daptomycin, polymyxin B, colistin, DCAP (2-((3-(3,6-dichloro-9H-carbazol-9-yl)-2-hydroxypropyl)amino)-2-(hydroxymethyl)propane-1,3-diol), an analog of DCAP, or a combination thereof.

43. The conjugate of claim 27, wherein the target bacterial cell comprises a Gram negative bacteria, and the polymer-drug conjugate interacts with an outer membrane of the Gram negative bacteria and a cell wall of the Gram negative bacteria.

44. The conjugate of claim 43, wherein the polymer-drug conjugate is an antibacterial agent-MCP conjugate comprising an antibacterial agent conjugated to a metal-chelating polymer (MCP).

45. The conjugate of claim 44, wherein the MCP has a molecular weight of from 4000 Da to 500 kDa, such as a molecular weight of from 50 kDa to 100 kDa.

46. The conjugate of any one of claims 44-45, wherein the MCP comprises a synthetic polymer.

47. The conjugate of any one of claims 44-46, wherein the MCP comprises a biopolymer.

48. The conjugate of claim 47, wherein the MCP comprises a polysaccharide.

49. The conjugate of any one of claims 44-48, wherein the MCP comprises a polymer bearing multiple carboxylic acid moieties, such as a poly(meth)acrylic acid or a copolymer thereof (e.g., poly(acrylic acid-co-maleic acid) (PAM)), a polyamine polymer, such as polyethyleneimine (PEI) or a copolymer thereof, a polymer bearing multiple iminodiacetic acid (IDA) moieties, such as a polymer derived from glycidyl methacrylate (GMA) and then modified to include modified to include IDA moieties, or a polysaccharide, such as chitosan, alginate, pectin, cellulose, glycogen, chitin, starch such as potato starch, glycosaminoglycans such as chondroitin, chondroitin sulfate, dermatan sulfate, hyaluronic acid, and keratan sulfate; aminated dextrans including DEAE-dextran; aminated starch, aminated glycogen, aminated cellulose, aminated pectin, and salts, complexes, derivatives and mixtures thereof.

50. The conjugate of any one of claims 44-49, wherein the antibacterial agent is covalently bound to the MCP via a non-cleavable linker.

51. The conjugate of any one of claims 44-49, wherein the antibacterial agent is covalently bound to the MCP optionally via a cleavable linker.

52. The conjugate of any one of claims 44-51, wherein the MCP is a negatively charged polymer.

53. The conjugate of any one of claims 44-52, wherein the antibacterial agent is an antibiotic that is indicated for use against a Gram positive bacterial infection.

54. The conjugate of any one of claims 44-53, wherein the antibacterial agent is an antibiotic that is not indicated for use against a Gram negative bacterial infection.

55. The conjugate of any one of claims 44-54, wherein the antibacterial agent comprises a glycopeptide antibiotic, such as vancomycin, teicoplanin, telavancin, oritavancin, dalbavancin, bleomycin, ramoplanin, or decaplanin.

56. The conjugate of any one of claims 44-55, wherein the antibacterial agent comprises a large beta-lactam antibiotic (i.e., a beta-lactam antibiotic that is not indicated for use against a Gram negative bacterial infection), such as penicillin.

57. The conjugate of any one of claims 44-56, wherein the antibacterial agent comprises a macrolide antibiotic, such as erythromycin, clarithromycin, azithromycin, roxithromycin, telithromycin, fidaxomicin, oleandomycin, methymycin, or tylosin.

58. The conjugate of any one of claims 44-57, wherein the antibacterial agent comprises a lipopeptide antibiotic, such as daptomycin, polymyxins (colistin and polymyxin B), surfactin, iturin, or fengycin; or a lincosamide antibiotic, such as clindamycin.

59. The conjugate of any one of claims 44-58, further comprising conjugating a second antibacterial agent to the MCP.

60. The conjugate of claim 59, wherein the second antibacterial agent comprises an antibiotic that is not indicated for use against both a Gram negative bacterial infection and a Gram positive bacterial infection (e.g., a broad spectrum antibiotic), and wherein the second antibacterial agent is covalently bound to the MCP optionally via a cleavable linker.

61. The conjugate of any one of claims 59-60, wherein the second antibacterial agent comprises doxycycline, minocycline, an aminoglycoside antibiotic (other than streptomycin), ampicillin, amoxicillin/clavulanic acid (Augmentin), azithromycin, a carbapenem antibiotic (e.g., imipenem), piperacillin/tazobactam, a quinolone antibiotic (e.g., ciprofloxacin), a tetracycline antibiotic (other than sarecycline), chloramphenicol, ticarcillin, trimethoprim/sulfamethoxazole (Bactrim), ofloxacin, or a combination thereof.

62. The conjugate of any one of claims 44-61, wherein the antibacterial agent-MCP conjugate comprises one or more covalently modified monomers defined by Formula I 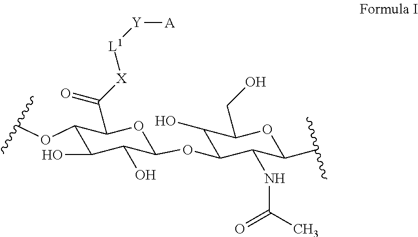
wherein
A is an antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

63. The conjugate of claim 62, wherein the linking group comprises a moiety formed by the chemical reaction of two click motifs.

64. The conjugate of any one of claims 44-63, wherein the antibacterial agent-MCP conjugate comprises a random copolymer defined by Formula II below 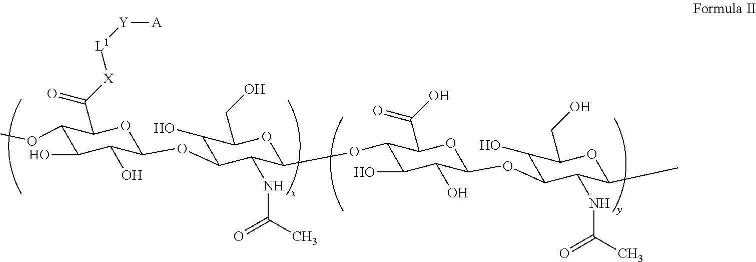
wherein
x and y are each independently integers from 1 to 2500, wherein x+y is no more than 2500, and wherein x and y represent the relative portion of each monomer within the random copolymer;
A is an antibacterial agent;
Y is absent, or is chosen from —O(C═O)—, —C(═O)—, —S(O)ₓ—, —C(═O)R^{a}C(═O)—, —NR^{a}C(═O)—, —NR^{a}C(═O)R^{a}C(═O)—, or —C(═O)NR^{a}C(═O)—;
L¹ is absent, or is a linking group; and
X is absent, or is chosen from NH, O, or S.

65. A method of treating or preventing a Gram negative bacterial infection in a subject, the method comprising administering to the subject a therapeutically or prophylactically effective amount of the polymer-drug conjugate defined by any one of claims 43-64.

66. The method of claim 65, wherein the Gram negative bacterial infection is caused by a bacteria of one of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella.

67. A method of treating or preventing a Gram positive bacterial infection in a subject, the method comprising administering to the subject a therapeutically or prophylactically effective amount of the polymer-drug conjugate defined by any one of claims 28-42.

68. A method of providing broad spectrum antibiotic therapy to a subject in need thereof to treat or prevent bacterial infection, the method comprising administering to the subject a therapeutically or prophylactically effective amount of the polymer-drug conjugate defined by any one of claims 26-64.

69. A method of treating or preventing a bacterial infection in a subject comprising a small colony variant, the method comprising administering to the subject a therapeutically or prophylactically effective amount of the polymer-drug conjugate defined by any one of claims 26-64.

70. The method of claim 69, wherein the small colony variant comprises Staphylococcus aureus.

71. A method of sensitizing a Gram negative bacteria towards an antimicrobial agent, the method comprising contacting the Gram negative bacteria with the polymer-drug conjugate defined by any one of claims 26-64.

72. The method of claim 71, wherein the Gram negative bacteria comprises a bacteria of one of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella.

73. A medical device comprising a coating comprising the polymer-drug conjugate defined by any one of claims 26-64.

74. The medical device of claim 73, wherein the medical device comprises an implantable (e.g., indwelling) medical device.

75. The medical device of any one of claims 73-74, wherein the medical device is formed at least in part from a metal, a ceramic, a polymer, or a combination thereof.

76. The medical device of any one of claims 73-75, wherein the medical device comprises an orthopedic implant or device, a catheter, a suture (resorbable or non-resorbable), a cardiovascular device (e.g., a pacemaker, a stent), an endotracheal tube, a tracheotomy tube, chest tube, a vascular catheter, an urinary catheter, a nephrostomy tube, a biliary stent, a peritoneal catheter, an epidural catheter, a central nervous system catheter, a prosthetic valve, a gastric tube, an intestinal tube, or a drug-delivery implant.
